(19) United States
(12) Patent Application Publication
(10) Pub. No.: US 2023/0167405 A1
(43) Pub. Date: Jun. 1, 2023

(54) METHODS AND COMPOSITIONS OF PRO-ORGAN FORMATION ON PARTICLE SUBSTRATES AND USES THEREOF

(71) Applicant: Herophilus, Inc., San Francisco, CA (US)

(72) Inventors: Morgan M. Stanton, San Francisco, CA (US); Oliver Wueseke, San Francisco, CA (US)

(21) Appl. No.: 17/861,108

(22) Filed: Jul. 8, 2022

Related U.S. Application Data
(63) Continuation of application No. PCT/US2021/012771, filed on Jan. 8, 2021.
(60) Provisional application No. 62/959,043, filed on Jan. 9, 2020.

(51) Int. Cl. C12N 5/0793; G01N 33/50; C12N 13/00

(52) U.S. Cl. CPC C12N 5/0619; G01N 33/5058; C12N 13/00; C12N 2513/00; C12N 2533/52; C12N 2533/54; C12N 2533/76; C12N 2506/45; C12N 2533/30; C12N 2531/00; G01N 2500/10

(57) ABSTRACT

Provided herein are compositions and methods describing the generation and use of in vitro pro-organs using microparticle scaffolds.

### CROSS REFERENCE

This application is a continuation of International Application No. PCT/US2021/012771, filed Jan. 8, 2021, which claims priority to U.S. Provisional Patent Application No. 62/959,043, filed on Jan. 9, 2020, both are incorporated herein by reference in their entirety for all purposes.

### BACKGROUND

Pro-organs are in vitro grown, three-dimensional tissue models that attempt to replicate complex biological systems for use in drug screening and disease modeling. Unlike a tissue grown in its native in vivo location, in vitro grown tissues lack the biochemical and biophysical signals, cell and cell-matrix interactions, and interplay among various cell types which are together important for a tissue's normal activity. Consequently, these pro-organs are unable to faithfully replicate complex biological systems which is needed for use in reliable drug screening and disease modeling.

### SUMMARY

The present invention addresses this need. Accordingly, the present disclosure provides neural pro-organs (i.e., neuroids) having properties that approximate complex neurological systems and are useful in drug screening and neural disease modeling.

An aspect of the present disclosure is a set of neuroids in which each neuroid in the set comprises one or more internalized microparticles.

In embodiments, at least one cell of each neuroid in the set expresses a neural marker, e.g., a cortical marker, hippocampal marker, cerebellar marker, retinal marker, midbrain marker, spinal cord marker, or brain stem marker. In embodiments, the cortical marker is FOXG1.

In embodiments, the one or more internalized microparticles comprises a polymer, glass, hydrogel, plastic, silica, ceramic, or magnetic substance; the one or more internalized microparticles is less than 500 μm in diameter; and/or the one or more internalized microparticles is greater than 2 μm in diameter.

In embodiments, the one or more internalized microparticles comprises a polymer comprising polystyrene (PS). The PS-comprising microparticles may be coated with polydopamine (PDA), which serves as a linker between the PS-comprising microparticles and a functional agent. In embodiments, the PS-comprising microparticle may be coupled to one or more functional agents. The functional agents are selected from fibronectin, laminin, collagen IV, and MATRIGEL. In embodiments, the functional agent is fibronectin. Alternately, the PS-comprising microparticle may lack a functional agent.

In embodiments, the one or more internalized microparticles comprises a hydrogel comprising agarose. The agarose-comprising microparticle may be coated with protein A or PDA, which serves as a linker between the agarose-comprising microparticle and a functional agent. In embodiments, the agarose-comprising microparticles may be coupled to one or more functional agents. The functional agents are selected from fibronectin, laminin, collagen IV, and MATRIGEL. Alternately, the agarose-comprising microparticles may lack a functional agent. In embodiments, at least one neuroid in the set further comprises at least one additional microparticle comprising MATRIGEL as a functional agent.

In embodiments, the one or more internalized microparticles comprises a magnetic substance. In embodiments, the microparticle comprising a magnetic substance may be coated with protein A or PDA, which serves as a linker between the microparticle comprising a magnetic substance and a functional agent. In embodiments, the microparticle comprising a magnetic substance may be coupled to one or more functional agents. The functional agents are selected from fibronectin, laminin, collagen IV, and MATRIGEL. Alternately, the microparticle comprising a magnetic substance may lack a functional agent.

In embodiments, the one or more internalized microparticles comprises glass. The glass-comprising microparticle may be coated with protein A or PDA, which serves as a linker between the glass-comprising microparticle and a functional agent. In embodiments, the glass-comprising microparticle may be coupled to one or more functional agents. The functional agents are selected from fibronectin, laminin, collagen IV, and MATRIGEL. Alternately, the glass-comprising microparticle may lack a functional agent.

In embodiments, the one or more internalized microparticles comprises silica, e.g., mesoporous silica. The mesoporous silica-comprising microparticle may be coated with protein A or PDA, which serves as a linker between the mesoporous silica-comprising microparticle and a functional agent. In embodiments, the mesoporous silica-comprising microparticle may be coupled to one or more functional agents. The functional agents are selected from fibronectin, laminin, collagen IV, and MATRIGEL. Alternately, the mesoporous silica microparticle may lack a functional agent.

In embodiments, the one or more internalized microparticles in a neuroid is coated on its surface by one or more functional agents and/or the one or more internalized microparticles encapsulates one or more functional agents. The one or more functional agents may modulate cell adhesion, cell growth, cell proliferation, cell organization, cell differentiation, cell repair, or cell regeneration in the neuroid.

In embodiments, a neuroid in the set further comprises at least one additional microparticle comprising one or more functional agents selected from a polypeptide, a polysaccharide, a small molecule, a nucleic acid, an imaging agent, MATRIGEL, hydrogel, and a sensor, or any combination thereof.

In embodiments, at least one neuroid in a set comprises a concentration gradient for the functional agent; the concentration gradient originates at an internalized microparticle.

In embodiments, at least one neuroid in the set is capable of generating neuronal activity, e.g., spontaneous neuronal activity, which may be oscillatory.

In embodiments, at least one neuroid in the set comprises a functional neural circuit between two cells in a neuroid.

In embodiments, at least one neuroid in the set are in fluid, physical, synaptic, and/or electrical communication with another neuroid in the set, e.g., which forms a functional neural circuit between the two neuroids.

In embodiments, a set of neuroids comprises at least 2, 3, 4, 5, 6, 7, 8, 9, 10, 50, 100, 300, 500, 1,000, 10,000, 100,000, or 1,000,000 neuroids. As examples, the set comprises 96, 384, and 1536 neuroids.

In embodiments, neuroids in a set are spatially dispersed in a two-dimensional array.

In embodiments, neuroids in a set are cultured in a cavity or microwell in a culture vessel; the cavities or microwells provide a consistent three-dimensional shape and size among neuroids in the set.

In embodiments, a plurality of neuroids in the set comprises fusion neuroids including two or more previously-distinct neuroids.

In embodiments, neuroids in the set comprise a fusion and/or contact between a neuroid and a non-neuroid tissue, e.g., a native neural tissue, a blood vessel tissue, and glial tissue, including microglia.

In embodiments, a first neuroid in the set comprises cells from a first cell line and a second neuroid in the set comprises cells from a second cell line, with the first cell line being different from the second cell line.

In embodiments, a plurality of neuroids in the set have a substantially similar phenotype. Alternately, at least one neuroid in the set comprises a phenotype of a neurological condition. The phenotype may relate to one or more of: size/shape of the neuroid, cell type, viability, number of cells, electrical activity, calcium activity, RNA expression, protein expression, genotype, or epigenomic type.

In embodiments, at least one neuroid in the set is derived from cells obtained from a subject having a neurological condition. A plurality of cells in a neuroid may comprise a genetic modification or mutation associated with a neurological condition.

In embodiments, at least one neuroid in the set comprises a total volume of internalized microparticles that is less than one third of the total volume of the neuroid.

In embodiments, the one or more internalized microparticles provides an at least 10% increase in the expression of a neural marker, e.g., a cortical marker, by cells of the one or more neuroids. The increase is relative to a neuroid prepared in an identical manner and with identical cells but that does not comprise the one or more internalized microparticles. In embodiments, the cortical marker may be FOXG1.

In embodiments, the one or more internalized microparticles provides an at least 20% increase in size of the one or more neuroids. The increase is relative to a neuroid that is prepared in an identical manner and with identical cell types but that does not comprise the one or more internalized microparticles.

In embodiments, the one or more internalized microparticles provides an at least 20% increase in volume of the one or more neuroids. The increase is relative to a neuroid that is prepared in an identical manner and with identical cell types but that does not comprise the one or more internalized microparticles.

In embodiments, the one or more internalized microparticles are coupled to one or more cells expressing a factor capable of promoting neuroid growth or patterning.

Another aspect of the present disclosure is a method of producing a neuroid comprising at least one internalized microparticle. The method comprises (a) providing a plurality of cells in a culture vessel, with at least one cell being a stem cell, e.g., an induced pluripotent stem cell (iPSC); (b) contacting the plurality of cells in the culture vessel with one or more microparticles; and (c) culturing the plurality of cells with the one or more microparticles. In embodiments, the one or more microparticles comprises at least one functional agent. In embodiments, the plurality of cells is provided to a cell culture vessel comprising the one or more microparticles. Alternately, the one or more microparticles is added to a cell culture vessel comprising the plurality of cells; the plurality of cells may have formed a cell aggregate before adding the one or more microparticles.

In embodiments, the method further comprises overlaying the plurality of cells and the one or more microparticles with a biocompatible matrix. The method may further comprise aggregating the plurality of cells and the one or more microparticles by co-incubation, centrifugation, or co-agitation.

In embodiments, the plurality of cells is co-incubated with the one or more microparticles for at least 12 hours; culturing the plurality of cells with the one or more microparticles occurs for up to 8 hours, up to 16 hours, up to 24 hours, up to 2 days, up to 3 days, up to 7 days, or up to 10 days; and/or culturing the neuroid comprising at least one internalized microparticle for at least 3 days, at least 7 days, at least 10 days, at least 14 days, at least 20 days, at least 30 days, at least 90 days, at least 200 days, at least 350 days or at least 500 days.

In embodiments, culturing the neuroid comprises positioning the neuroid at an air-liquid interface.

In embodiments, a surface of the culture vessel comprises one or more cavities or microwells which provide consistent three-dimensional shape and size among neuroids. In embodiments, the culture vessel comprises a low-adhesion surface, e.g., an ultra-low adhesion plate, which helps prevent binding of cells to the vessel surface. In embodiments, a surface of the culture vessel lacks a coating, e.g., with a biocompatible matrix.

In embodiments, the method further comprises isolating a differentiated neural cell of interest from the neuroid, e.g., the cell of interest isolated based on the presence or absence of a neural marker. The neural marker may be a cortical marker, e.g., FOXG1.

In embodiments, the one or more microparticles comprises a polymer, glass, hydrogel, plastic, silica, ceramic, or magnetic substance.

In embodiments, a plurality of the one or more microparticles comprises a functional agent.

In embodiments, the one or more microparticles are coated with or coupled to one or more functional agents selected from fibronectin, laminin, collagen IV, and MATRIGEL. The coating may be by co-incubation and/or the coupling may be by chemical cross-linking, co-incubation, co-agitation, or centrifugation. In embodiments, the co-incubating of the one or more microparticles with the functional agent occurs for at least 10 minutes, for at least 30 minutes, for at least 1 hour, for at least 4 hours, for at least 8 hours, for at least 12 hours, for at least 16 hours, or for at least 24 hours.

Another aspect of the present disclosure is a method of producing a neuroid comprising at least one internalized microparticle. The method comprises steps of (i) providing a plurality of cells in a culture vessel, wherein at least one cell is a stem cell; (ii) contacting the plurality of cells in the culture vessel with one or more microparticles lacking a functional agent; (iii) culturing the plurality of cells with the one or more microparticles lacking a functional agent; (iv) contacting the one or more microparticles with MATRIGEL, thereby allowing the microparticles to absorb the MATRIGEL; and (v) further culturing the plurality of cells with the one or more microparticles that have absorbed MATRIGEL to produce a neuroid comprising at least one internalized MATRIGEL-absorbed microparticle.

In embodiments, step (iii) occurs for up to 8 hours, up to 16 hours, up to 24 hours, up to 2 days, up to 3 days, up to 7 days, or up to 10 days and/or step (iv) occurs after completion of step (iii). In embodiments, the method further comprises culturing the neuroid comprising at least one internalized MATRIGEL-absorbed microparticle for at least 3 days, at least 7 days, at least 10 days, at least 14 days, at least 20 days, at least 30 days, at least 90 days, at least 200 days, at least 350 days or at least 500 days. The culturing of the plurality of cells with the one or more microparticles lacking a functional agent delays ingestion of the microparticles which provides improved rosette (e.g., neuronal rosette) formation and area in the neuroid once contacted the one or more microparticles with MATRIGEL. The improved rosette formation and area is relative to a neuroid lacking a microparticle comprising MATRIGEL.

Another aspect of the present disclosure is a method of producing a neuroid comprising at least one internalized microparticle. The method comprises steps of (I) providing a plurality of cells in a culture vessel, wherein at least one cell is a stem cell; (II) culturing the plurality of cells in the absence of a microparticle until a developing neuroid has formed or begun to form; (III) adding one or more microparticles comprising MATRIGEL and allowing the developing neuroid to internalize the microparticles comprising MATRIGEL; and (IV) further culturing the developing neuroid to produce a neuroid comprising at least one internalized MATRIGEL-absorbed microparticle.

In embodiments, step (II) occurs for up to 8 hours, up to 16 hours, up to 24 hours, up to 2 days, up to 3 days, up to 7 days, or up to 10 days and/or step (III) occurs after completion of step (II). In embodiments, the method further comprises culturing the neuroid comprising at least one internalized MATRIGEL-absorbed microparticle for at least 3 days, at least 7 days, at least 10 days, at least 14 days, at least 20 days, at least 30 days, at least 90 days, at least 200 days, at least 350 days or at least 500 days. The culturing of the plurality of cells in the absence of a microparticle provides improved rosette (e.g., neuronal rosette) formation and area in the neuroid once contacted the one or more microparticles with MATRIGEL. The improved rosette formation and area is relative to a neuroid lacking a microparticle comprising MATRIGEL.

Yet another aspect of the present disclosure is a neuroid produced by any herein-disclosed method.

In an aspect, the present disclosure provides a method for identifying an agent capable of producing a phenotypic change in a neuroid or in a cell thereof. The method comprises contacting an agent with a neuroid of the present disclosure. When a phenotypic change is detected in the neuroid or by a cell thereof, the agent is identified as being capable of producing a phenotypic change in a neuroid or a cell thereof, and possibly useful as a therapeutic, e.g., for treating a neurological disease.

In embodiments, the phenotypic change is a modification in the neuroid's longest dimension, cell type, shape, viability, number of cells, oscillatory electrical activity, calcium activity, signaling pathway activation, protein-protein interactions, phosphorylation, ubiquitination, sumolyation, RNA expression, protein expression, genotype, and/or epigenomic type.

In another aspect, the present disclosure provides an agent for producing a phenotypic change in a neuroid or in a cell thereof. The agent is identified by a herein-disclosed method. An agent for producing a phenotypic change in a neuroid or a cell thereof may be used in a method of drug discovery for treating a neurological disease.

An aspect of the present disclosure is a method of manipulating one or more neuroids in a culture vessel. The method including (a) providing one or more neuroids having at least one internalized microparticle comprising a magnetic substance; and (b) applying an external magnetic field to the one or more neuroids. In embodiments, the external magnetic field is provided by a magnet that does not contact a liquid medium in the culture vessel. Manipulating the one or more neuroids in the culture vessel may comprise adjusting the position and/or orientation of the one or more neuroids by the applied external magnetic field, restricting motion of the one or more neuroids in its location in the culture vessel, and/or separating the one or more neuroids from a surface of the culture vessel.

In embodiments, the method further comprises analyzing the one or more neuroids by microscopy.

It shall be understood that different aspects and/or embodiments of the invention can be appreciated individually, collectively, or in combination with each other. Various aspects and/or embodiments of the invention described herein may be applied to any of the uses set forth below and in other methods for making and using neuroids. Any description herein concerning a specific composition and/or method applies to and may be used for any other specific composition and/or method as disclosed herein. Additionally, any composition disclosed herein is applicable to any herein-disclosed method. In other words, any aspect or embodiment disclosed herein can be combined with any other aspect or embodiment as disclosed herein.

### BRIEF DESCRIPTION OF THE DRAWINGS

The patent or application file contains at least one drawing executed in color. Copies of this patent or patent application publication with color drawings will be provided by the Office upon request and payment of the necessary fee. The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A to FIG. 1D show a comparison of optimal and non-optimal characteristics of neuroids. FIG. 1A shows a non-optimal neuroid with large rosette (e.g., neuronal rosette) formation, but no FOXG1 expression. FIG. 1B shows a non-optimal neuroid with small or no rosette formation and no FOXG1 expression. FIG. 1C shows an optimal set of characteristics of a neuroid with large rosette formation and FOXG1 expression. FIG. 1D shows a non-optimal neuroid with small or no rosette formation and a small amount of FOXG1 expression.

FIG. 2A to FIG. 2C show bright field images of control neuroids cultured without microparticles and neuroids cultured with incorporated microparticles.

FIG. 3 shows the quantification of rosette formation in neuroids that have incorporated polystyrene particles; a comparison between four different cell lines is shown.

FIG. 4A to FIG. 4D show immunofluorescence images of FOXG1 and DAPI staining of neuroid slices from four different cell lines with or without polystyrene microparticles coated with fibronectin, laminin, or collagen IV.

FIG. 5 shows the quantification of rosette formation in neuroids from two different cell lines, grown with agarose microparticles coated with fibronectin or laminin.

FIG. 6A and FIG. 6B show immunofluorescence images of FOXG1 and DAPI staining of neuroid slices from two different cell lines with or without with agarose microparticles modified with fibronectin or laminin.

FIG. 7 shows images of neuroid maturation with or without magnetic microparticles over a 39-day culture period.

FIG. 8 shows manipulation of neuroids having internalized magnetic microparticles by an applied external magnetic field.

FIG. 9 shows fluorescent images identifying protein expression by neuroids with or without internalized magnetic microparticles.

### DETAILED DESCRIPTION

The present disclosure is based, in part, on the discovery of neural pro-organs (i.e., neuroids) having properties that approximate complex biological systems, e.g., neural systems.

### Introduction

The neural pro-organs (i.e., neuroids) of the present disclosure exhibit features, e.g., molecular markers or electrical activity, that are found in the mammalian nervous system. Also, neuroids of the present disclosure may have features that are specific to subsets of the mammalian nervous system, e.g., particular regions of the brain. The methods of the present disclosure can produce sets of neuroids in which each neuroid in the set has nearly identical features; since the methods are scalable, large quantities of consistent neuroids can be produced. Together, these allow for reproducible and/or well-controlled experiments. In some cases, the neuroids are derived from cells obtained from a patient having a neurological condition; in other cases, cells in a neuroid are genetically modified to include mutations associated with a neurological condition. The neuroids may be amenable to real-time image analysis and parallel cellular-biochemical analyses, with minimal handling steps; these, allow uses in high-throughput drug screening assays or in the development of drug therapies. Accordingly, the neuroids of the present disclosure allow for improved, robust, and consistent applications as neurological disease models and as drug discovery tools.

### Pro-organs

Pro-organs are three-dimensional (3D) culture models that replicate complex biological systems in vitro. A pro-organ is an aggregation of cells that make functional contacts with other cells in the aggregate. A pro-organ can be thought of as in vitro produced, three-dimensional tissue, which resembles the structure, marker expression, and/or function of an in vivo organ. The pro-organ retains at least one characteristic of an in vivo tissue, e.g., prolonged tissue expansion with proliferation, multilineage differentiation, and/or recapitulation of cellular and tissue ultrastructure. Since a pro-organ may be considered to be a miniaturized and simplified version of an in vivo organ, it is useful for studying disease biology and for developing novel drug candidates.

A pro-organ can comprise a variety of cell types. In some cases, the pro-organ can comprise or be derived from one or more of pluripotent stem cells, multipotent stem cells, progenitor cells, terminally differentiated cells, immortalized cell lines, or primary cells. A pro-organ can comprise or be derived from ectodermal, endodermal, or mesodermal progenitor cells. A pro-organ can comprise or be derived from induced pluripotent stem cells (iPSCs), e.g., patient-specific iPSC lines that are generated from tissue samples, thereby allowing the formation of patient-specific pro-organs. A pro-organ can comprise or be derived from cells that have undergone a degree of differentiation. A pro-organ can comprise cells that have undergone terminal differentiation. A pro-organ can include without limitation, embryoid bodies and embryoid-like bodies, or similar structures. An embryoid body, and the like, is a plurality of cells containing pluripotent or multipotent stem cells formed into a three-dimensional sphere or another three-dimensional shape. A pro-organ may comprise a specific cell type or have features of specific cell types; as examples, a pro-organ, identified by its main constituent, may be a neural pro-organ, retinal pro-organ, cardiac pro-organ, or epithelial pro-organ.

### Neuroids

A specific type of pro-organ relevant to the present disclosure is a neural pro-organ. Neural pro-organs, referred to herein as neuroids, comprise or are derived from neural cells, neural stem cells or neural progenitors, cells expressing neural markers, and/or cells having neural functions/activity.

Several limitations exist with current neural pro-organ technologies that prevent full realization of their value as neurological disease models and drug discovery tools. Limitations including low brain region specific reproducibility (e.g., defects in patterning) and formation of non-native tissue cytoarchitecture (e.g., defects in structure). In certain cases, multiple pro-organs generated from a patient's iPSC line can vary widely in cytoarchitecture, cell type diversity, and genetic profile, making it difficult to collect reproducible phenotypic profiling and/or drug efficacy data. Different methodologies in neural pro-organ culture have been explored to solve these problems. However, these solutions have provided limited improvement in neural pro-organ reproducibility both within and across iPSC lines. Recently, biomaterials internalized within neuronal pro-organs have been tested with natural fibers (poly lactic-co-glycolic acid, sea sponge, or cellulose fibers) and were shown to increase cell surface area, but the effects were transient and not reproducible.

In contrast, neuroids of the present disclosure properly model native nervous system tissue, express a range of neural markers (including brain region-specific genetic and molecular markers), and comprise cellular diversity and cytoarchitecture consistent with in vivo nervous system tissue. Also, neuroids of the present disclosure possess functions/activity that approximate in vivo neural system tissue and/or organs. Consequently, neuroids of the present disclosure overcome defects in patterning and/or structure, as examples, that are observed in current neural pro-organs.

The neuroids of the present disclosure overcome these defects, at least in part, by comprising internalized microparticles having attributes that promote neuroid formation, development, and activity. Indeed, an aspect of the present disclosure is a set of neuroids in which each neuroid in the set comprises one or more internalized microparticles.

Cells in a set of neuroids express one or more neural markers. By neural marker is meant any protein (or a polynucleotide encoding the same) for which expression is associated with a neural cell fate, function, and/or activity.

A neuroid can express one or more neural markers characteristic of one or more regions of the brain. In embodiments, at least one cell of each neuroid expresses a neural marker, e.g., a cortical marker, hippocampal marker, cerebellar marker, retinal marker, midbrain marker, spinal cord marker, and brain stem marker, and/or markers of specific cell types found in the mammalian nervous system, e.g., dopaminergic neurons, granular neurons, GABAergic neurons, astrocytes, oligodendrocytes, microglia, vascular cell. In embodiments, the cortical marker is FOXG1. In some cases, a neuroid contains cells representative of more than one brain region. For example, a neuroid may include cortical features and cerebellar features or a neuroid may have brainstem features and spinal cord features. In some cases, a neuroid has distinct domains where each domain comprises cells representing one brain region. This configuration is possible, at least, in fusion neuroids, which are formed from two or more previously-distinct neuroids, e.g., each comprising a different brain region-specific cell type. Also, a neuroid may be contacted with and/or fused with a non-neuroid tissue, e.g., a native neural tissue, a blood vessel tissue, and glial tissue, including microglia.

Illustrative cerebellar markers include, but are not limited to, ATOH1, PAX6, SOX2, LHX2, and GRID2. Illustrative markers of dopaminergic neurons include, but are not limited to, tyrosine hydroxylase, vesicular monoamine transporter 2 (VMAT2), dopamine active transporter (DAT) and Dopamine receptor D2 (D2R). Illustrative cortical markers include, but are not limited to, doublecortin, NeuN, FOXG1, FOXP2, CNTN4, and TBR1. Illustrative retinal markers include, but are not limited to, retina specific Guanylate Cyclases (GUY2D, GUY2F), Retina and Anterior Neural Fold Homeobox (RAX), and retina specific Amine Oxidase, Copper Containing 2 (AOC2). Illustrative granular neuron markers include, but are not limited to, SOX2, NeuroD1, DCX, EMX2, FOXG1, and PROX1. Illustrative brain stem markers include, but are not limited to, FGF8, INSM1, GATA2, ASCL1, GATA3. Illustrative spinal cord markers include, but are not limited to, homeobox genes including but not limited to HOXA1, A2, A3, B4, A5, C8, or D13. Illustrative GABAergic markers include, but are not limited to, NKCC1 or KCC2. Illustrative astrocytic markers include, but are not limited to, GFAP. Illustrative oliogodendrocytic markers include, but are not limited to, OLIG2 or MBP. Illustrative microglia markers include, but are not limited to, AIF1 or CD4. Illustrative vascular cell markers include, but are not limited to, NOS3.

A neuroid of the present disclosure can have features of specific regions of the mammalian nervous system and can be used as a model for those specific regions of the mammalian nervous system. For example, the neuroid may serve as a cortical neuroid by expressing neural markers characteristic of cortical neurons (e.g., FOXG1), by forming neural circuits akin to circuits in the mammalian cortex, and/or by acquiring cell shapes and an overall structure similar to cortical tissues. Other neuroids may serves as forebrain neuroids, ventral forebrain neuroids, midbrain neuroids, early mid-brain neuroids, late mid-brain neuroids, brain-stem neuroids, spinal-cord neuroids, or retinal neuroids, and a combination thereof.

In embodiments, at least one neuroid comprises a functional neural circuit between two cells in a neuroid. Additionally, in embodiments, a functional neural circuit can form between cells of two neuroids. A functional neural circuit is a connection between cells of a neuroid or between cells of two neuroids that permits transmission of information from one cell to another cell. The connection may be via synapses or synapse-like domains. In embodiments, a plurality of neuroids in a set are in fluid, physical, synaptic, and/or electrical communication with other neuroids in the set; this connections forms, at least a part of, a functional neural circuit between two neuroids. Functional neural circuit may be characterized by electrical activity patterns within a network of cells/neuroids. In embodiments, at least one neuroid in the set is capable of generating neuronal activity, e.g., spontaneous neuronal activity, which may be oscillatory.

A neuroid may comprise or be derived from cells of one cell line (e.g., cell type). A neuroid may comprise cells from a first cell line and cells from a second cell line, with the first cell line being a different cell type from the second cell line.

In embodiments, a plurality of neuroids in a set comprise or are derived from one cell line and/or cell type. For example, a first neuroid and a second neuroid comprises cells from a first cell line. The neuroids in these sets may have a substantially similar phenotype. The phenotype may relate to one or more of: size/shape of the neuroid, cell type, viability, number of cells, electrical activity, calcium activity, RNA expression, protein expression, genotype, or epigenomic type.

In embodiments, a plurality of neuroids in a set comprise or are derived from different cell types. For example, a first neuroid comprises cells from a first cell line and a second neuroid in the set comprises cells from a second cell line, with the first cell line being different from the second cell line. The neuroids in these sets may have certain phenotypes that differ. The phenotype may relate to one or more of: size/shape of the neuroid, cell type, viability, number of cells, electrical activity, calcium activity, RNA expression, protein expression, genotype, or epigenomic type.

In embodiments, at least one neuroid in the set comprises a phenotype of a neurological condition.

In embodiments, at least one neuroid in the set is derived from cells obtained from a subject having a neurological condition. In embodiments, a plurality of cells in at least one neuroid in the set comprises a genetic modification or mutation associated with a neurological condition (either native to the cell or created in the cell, e.g., by gene editing).

In embodiments, a plurality of neuroids in a set may comprise fusion neuroids which include two or more previously-distinct neuroids. As examples, a fusion neuroid comprises two neuroids each comprising or derived from cells of the same line; a fusion neuroid comprises two neuroids each comprising or derived from cells of a different line; and a fusion neuroid comprises three neuroids each comprising or derived from cells of different lines. Such fusion neuroids may approximate junctions/connections between different regions of the mammalian nervous system.

In embodiments, neuroids in a set comprise a fusion and/or contact between a neuroid and a non-neuroid tissue, e.g., a native neural tissue, a blood vessel tissue, and glial tissue, including microglia.

A neuroid grows in three dimensions. Thus, a neuroid has a 3-D shape, e.g., a spherical, spheroidal, or oblong shape. Alternately, a neuroid may resemble a flattened disc. In embodiments, neuroids are cultured in a cavity or microwell in a culture vessel. When neuroids are cultured in cavities or microwells of similar size and shape, the resulting neuroids will have consistent three-dimensional size and shape. Additionally, the resulting neuroid may have consistent cell densities. Together, such neuroids are standardized (at least in size, density, and/or shape) which provides for consistent and reproducible uses, e.g., in modeling and drug discovery.

In some cases, the various cell types within a neuroid can exhibit a spatial organization pattern. For example, cells in the neuroid can be organized along a dorsal-ventral axis (roof, alar, basal and floor plate) and/or along a rostral-caudal axis (telencephalon, diencephalon, mesencephalon, rhomboid encephalon, and spinal cord). In some cases, the neuroid of the present disclosure comprise both self-renewing progenitors and differentiated cell types that show migratory motion. In some cases, the neuroid comprises intermediate progenitors that could migrate outward towards region-specific stratified structures such as the medulla, the optic tectum and the cerebral cortex. In some cases, the neuroid recapitulate the spatial and temporal events leading to the formation of layered structures in the mammalian brain, e.g., stratified cortical-like tissues containing 1, 2, 3, 4, 5, or 6 layers. In some cases, the neuroid adopts a rostral-hypothalamic fate. In some cases, the neuroid adopts a regional fate (e.g., olfactory bulb, rostral and caudal cortices, cortical hem, and choroid plexus). In some cases, the neuroid adopts an adenohypophysis fate. In some cases, neuroid comprises features of outer radial glial cells.

In embodiments, neuroids in a set are spatially dispersed in a two-dimensional array. Such an array, at least, allows testing of neural circuit formation and/or consequences of connections between neuroids.

In embodiments, two adjacent neuroids are nearly touching. In some cases, two neuroids are grown or subsequently positioned to be in physical contact. Whether touching or nearly touching, a neuroid in the array may be in communication with at least one other neuroid. In some cases, a neuroid in an array (e.g., in a set) are in fluid, physical, synaptic, and/or electrical communication with another neuroid in the array (or set).

Alternately, two adjacent neuroids are physically separated in a two-dimensional array. Here, the effects of breaking or distancing natural connections (e.g., between brain regions) can be assayed. These assays may be useful for axon growth assays where neurons seek out and connect with their target cell.

The array of neuroids may be present on a culture vessel surface. Here, the array of neuroids are grown in situ on the vessel surface or placed on the vessel surface after, at least, initial growth. To enhance microscopic imaging (e.g., time-lapse imaging), preferably each neuroid in a two-dimensional array is located within a single focal plane.

Neuroids in a two-dimensional array may be present at varying densities. As examples, the density is at least one neuroid per cm² and at least thirty neuroids per cm². In some cases, the distance between the center of mass of adjacent neuroids in an array is from about 10 to about 5000 μm, preferably 10 to about 2000 μm, such that the array is suitable for high-throughput processing and imaging. In some cases, the array is compatible with liquid handling, automated liquid handling, high throughput screening, and/or micro-pipetting.

In embodiments, a set of neuroids comprises at least 2, 3, 4, 5, 6, 7, 8, 9, 10, 50, 100, 300, 500, 1,000, 10,000, 100,000, or 1,000,000 neuroids, or any number therebetween. As examples, the set comprises 96, 384, and 1536 neuroids.

### Neuroid Uses

Neuroids of the present disclosure allow for improved, robust, and consistent uses as neurological disease models and as drug discovery tools. The neuroids of the present disclosure may be regarded as miniaturized and/or simplified models of neural organs, including the brain (or regions thereof) and spinal cord.

The neuroids described herein can be used for drug discovery and drug refinement by phenotypic drug screening. Phenotypic drug screening differs from target-specific drug screening techniques: in target-specific drug screening, the focus is on the binding of a candidate molecule to a specific target; instead, in phenotypic drug screening the focus is on the effect that a target molecule has on a phenotype (of a cell, tissue, or pro-organ, i.e., neuroid). This use of neuroids includes the discovery and refinement of drugs that may induce or enhance repair, regeneration, protection, and disease prevention in brain and neural tissue.

The neuroids described herein can be used as disease models for investigating various diseases related to neural tissues. In embodiments, a neuroid comprises a phenotype of a neurological condition. This may be due, in part, from the neuroid being derived from cells obtained from a subject having a neurological condition and/or from neuroid being derived from cells comprising a genetic or epigenetic modification or mutation associated with a neurological condition. The genetic modification or mutation may have been created in a cell by gene editing or anther directed mutagenesis method. Neuroids comprising cells or derived from cells obtained from healthy individuals and/or from cell lines (and that lack any genetic modification or mutation) are useful for modeling and from drug discovery in healthy, normal nervous systems.

Neuroids of the present disclosure allow assays and analyses with high predictive value. The higher degree of maturation, cellular complexity, and hierarchical network function of the neuroids when coupled with the scalability of neuroid production readily enables high-throughput phenotypic screening. As an example, sets of healthy and/or disease-modelled neuroids can be subjected to simulated diseases (for example, hypoxia to induce stroke-like damage). Phenotypic read-outs include effects on tissue formation, electrical connectivity, cell death, and cell proliferation of the neuroid itself and/or of specific cell types within a neuroid. A phenotypic drug screen can allow the definition and validation of a variety of candidate drugs. This can provide a basis for subsequent compound screening for identifying compounds having regenerative, reparative, disease modifying, or protective biological activity, as examples.

The neuroids of the present disclosure serve as useful models and/or for drug screening of human neurological diseases, including stroke, brain inflammation disorders, neurodegenerative diseases (e.g., Parkinson's Disease and Alzheimer's Disease), neuroinflammatory diseases (e.g., multiple sclerosis), traumatic injury (e.g., brain-surgery-induced injury), channelopathy (e.g., epilepsy), and psychiatric diseases (including autism and schizophrenia). Neuroids of the present disclosure also are useful for drug safety screening to test, for instance, the potential of a substance to induce electrical disturbances (seizures), degeneration, cell death, or other cellular anomalies in neural tissue. Neuroids of the present disclosure can also be used in mechanism-of-action studies involving drugs, e.g., in preclinical trials running before or in parallel with clinical trials.

Neuroids derived from a patients' cells, are useful in personalized medicine uses. For example, a patient-derived neuroid can be tested with a variety of therapeutics to predict how the patient's nervous system would respond when the therapeutic is administered to the patient him/herself. Such therapeutics may include drugs (e.g., small molecules and chemotherapies), biologics (e.g., antibodies), physical agents (e.g., ionizing radiation), or gene-therapy-related treatments (e.g., RNAi and gene editing proteins), or a combination thereof.

The neuroids of the present disclosure may provide regenerative tissue for scientific or therapeutic purposes. For example, the neuroids may be injured to study recovery and regeneration under drug or biophysical (e.g., electrical conditioning) treatment. Alternatively, the neuroids may be constructed with specific brain functions, such as dopamine production and release to counter Parkinson's Disease. Further the neuroids may be connected to organs or used as machine-organ interfaces to enable control of enervated organs (e.g., control of skeletal muscle).

Neuroids can be used to study development/maturation of cells in a neuroid, development/maturation of the neuroid itself, or development/maturation of sets of neuroids. Maturation of neuronal cells can be measured based on morphology by optically assessing parameters such as dendritic arborization, axon elongation, total area of neuronal cell bodies, number of primary processes per neuron, total length of processes per neuron, number of branching points per primary process as well as density and size of synaptic puncta stained by synaptic markers (e.g., synapsin-1, synaptophysin, bassoon, PSD95, and Homer). Moreover, general neuronal maturation and differentiation can be assessed by measuring expression of marker proteins (e.g., FOXG1, MAP2, TUJ-1, NeuN, Tau, PSA-NCAM, and SYN-1), alone or in combination using FACS analysis, immunoblotting, fluorescence microscopy imaging, or patch clamping. Maturation and differentiation of neuronal subtypes can further be tested by measuring expression of specific proteins. For excitatory neuronal cells this includes staining for VGLUT1/2 and GRIA1/2/3/4, as examples. Formation of a spatial organization pattern in a neuroid can be characterized, e.g., formation of distinct regions in a neuroid, such as cortical-like layers. Moreover, interactions between neuroids in a set can be studied, e.g., to better understand cell-cell contacts, including gap junction and synapse formation, as examples.

Neuroids may be assayed in combination with non-neural tissues, cells, and/or pro-organs. These combinations may approximate connections that nervous system cells/tissues make with other cells/tissues of the body. For example, assays relating to contacts between neural tissue enervating skeletal muscle (e.g., via nicotinergic nerve endings at skeletal neuromuscular junctions) can be conducted. For this, a neuroid can be co-cultured with bioengineered skeletal muscle (BSM) to study neuro-muscular junction development and activity. Also, co-cultures of neuroids with Engineered Heart Muscle (EHM) can be used to study interactions between the sympathetic nervous system and cardiac muscle cells; this model can then be used to study development of and treatments for arrhythmia. Neuroids can also be co-cultured with Engineered Skeletal Muscle (ESM), Engineered Liver Tissue (ELT), or Engineered Connective Tissue (ECT). In other embodiments, neuroid-tumor models are used to study tumor-brain interactions (e.g., tumor brain invasion and metastases spread) or neuroid-leukocyte infiltration models to study neuronal inflammation. These co-culture models represent an important step towards screening drug actions on multiple organ systems.

Neuroids of the present disclosure may be implanted into a living animal. This use may help repair damaged neural tissue, e.g., from trauma, a stroke, and a neurodegenerative disorder. It may contribute to neural development in an animal having an abnormal or deficient nervous system, e.g., due to an environmental cause or genetic mutation. An implanted neuroid may secrete factors that contribute to neuronal and non-neuronal activity, growth, and/or health of tissues in the region of implantation.

### Screening Assays

Neuroids of the present disclosure are useful in screening assays for quantitatively and qualitatively assessing neuroid development, maturation, functions, or perturbations thereof. The screening assays generally involve contacting neuroids with a candidate agent and determining a phenotypic effect on the neuroid, if at all.

An aspect of the present disclosure is a method for identifying an agent capable of producing a phenotypic change in a neuroid or in a cell thereof. The method comprises contacting an agent with a neuroid. When a phenotypic change is detected in one or more neuroids or by a cell thereof, the agent is identified as being capable of producing a phenotypic change in a neuroid or in a cell thereof.

In embodiments, the phenotypic change is a modification in the neuroid's longest dimension, cell type, shape, viability, number of cells, oscillatory electrical activity, calcium activity, signaling pathway activation, protein-protein interactions, phosphorylation, ubiquitination, sumolyation, RNA expression, protein expression, genotype, and/or epigenomic type.

Candidate agents of interest can be biologically active agents. In some cases, biologically active agents can encompass numerous chemical classes, primarily organic molecules, which may include organometallic molecules, inorganic molecules, genetic sequences. Neuroids of the present disclosure can be used to evaluate candidate drugs, select therapeutic antibodies and protein-based therapeutics, with preferred biological response functions. Candidate agents can comprise functional groups necessary for structural interaction with proteins, particularly hydrogen bonding, and typically include at least an amine, carbonyl, hydroxyl or carboxyl group, frequently at least two of the functional chemical groups. The candidate agents can comprise cyclical carbon or heterocyclic structures and/or aromatic or polyaromatic structures substituted with one or more of the above functional groups. Candidate agents can also be found among biomolecules, including peptides, polynucleotides, saccharides, fatty acids, steroids, purines, pyrimidines, derivatives, structural analogs or combinations thereof.

Candidate agents also include known pharmacologically active drugs. Compounds of interest can include chemotherapeutic agents, anti-inflammatory agents, hormones or hormone antagonists, ion channel modifiers, and neuroactive agents. Illustrative suitable pharmaceutical agents are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Cardiovascular Drugs; Vitamins, Dermatology; and Toxicology. The contents of which is incorporated herein by reference in its entirety.

A candidate agent can comprise samples of unknown content. Samples can include mixtures of naturally occurring compounds derived/extracted from biological sources (e.g., microbes, plants, and animals), environmental sources (e.g., soil, ground water, sea water, and mining waste), and manufacturing samples (e.g., intermediates and byproducts from preparation of pharmaceuticals).

Candidate agents can be obtained from a wide variety of sources including libraries of synthetic or natural compounds. The library-derived compounds can be tested individually on neuroids or they can be mixed into a composition before being tested. Alternatively, libraries of natural compounds in the form of bacterial, fungal, plant, and animal extracts are available or can be readily produced.

The candidate agents can be added to a culture medium comprising neuroids in a flow-through system, as a stream, intermittent or continuous, or alternatively, adding a bolus of the compound, singly or incrementally, to an otherwise static culture medium. The overall concentrations of the components of the culture medium should not change significantly with the addition of the bolus, or between the two solutions in a flow through method.

A plurality of assays may be run in parallel—with different agent concentrations—to obtain a differential response to the various concentrations. Typically, one of these concentrations serves as a negative control, e.g., at zero concentration or below the concentration of the candidate agent that can induce a detectable change in phenotype.)

A candidate agent may be a genetic agent, which refers to polynucleotides and analogs thereof. The introduction of a genetic agent to a neuroid can result in an alteration of the total genetic composition of the cell and/or gene expression by the cell and/or protein synthesis in the cell. Genetic agents can change the genome of a cell, generally through the integration of a nucleic acid sequence into a chromosome. The genetic agent may express components of a gene editing system, e.g., CRISPR and TALEN. Genetic changes may be transient, with the genetic agent not being integrated into the genome. Antisense oligonucleotides, as genetic agents, can affect the expression of proteins without changing the cell's genome, by interfering with the transcription or translation of mRNA. The effect of a genetic agent is to increase or decrease expression of one or more gene products in the cell.

Candidate agents may be provided to a neuroid along with a change in one or more environmental conditions, e.g., following stimulation with an agonist, change in temperature, change in oxygen levels, and following electric or mechanical stimulation.

An aspect of the present disclosure relates to agents capable of producing a phenotypic change in a neuroid or a cell thereof, e.g., to be used in a method of drug discovery for treating a neurological disease.

Phenotypic effects of a candidate agent on a neuroid can be assessed by monitoring multiple output parameters. Output parameters include morphological, functional, and/or genetic changes. These include changes in expression of cytoplasmic, cell surface, and/or secreted biomolecules; also, alterations in the types and amounts of biopolymers, e.g., polypeptides, polysaccharides, polynucleotides, and lipids. Expression of cell surface and secreted molecules is a useful parameter to be assayed, as these frequently mediate cell-cell communications and cell-effector responses. In embodiments, detecting phenotypic effects comprises analyzing neuroids by microscopy.

Live imaging, using microscopy, of cells may be enhanced in neuroids comprising cells that express a detectable marker.

Gene/protein expression changes in response to a candidate agent may be detected using any method well-known in the art. Examples include, fluorescently-labeled proteins, immunoassay techniques (including immunohistochemistry, Western/dot blots, and enzyme linked immunosorbance assay (ELISA)).

Methods of analysis at the single cell level can be of interest. Live imaging (including confocal or light microscopy), single cell gene expression or single cell RNA sequencing, calcium imaging, immunocytochemistry, patch-clamping, flow cytometry, and the like may be used.

Neuroids possessing electrical activity (e.g., capable of generating an electrical signal in response to external stimuli and capable of generating spontaneous electrical signals) can be assayed using electrophysiological recordings, using voltage-sensitive dyes (e.g., di-4-ANEPPS, di ANEPPS, and RH237), and/or calcium sensitive dyes (e.g., Fura-2 calcium imaging; Fluo-4 calcium imaging, GCaMP6 calcium imaging). Cells of a neuroid may comprise genetically-encoded voltage indicators (e.g., ASAP1, Archer). Neuronal-like activity can quantify/characterize the occurrence, intensity, frequency, duration, and patterns of electrical signals. Neuroids possessing electrical activity may have spontaneous activity, e.g., in the absence of contact with a candidate agent and/or by electrical stimulation, and/or may have induced activity, e.g., in response to contact with a candidate agent and/or by electrical stimulation.

Recordings of neuronal activity by neuroids can be conducted after about 3 days, after about 4 days, after about 5 days, after about 1 week, after about 2 weeks, after about 3 weeks, after about 4 weeks, after about 6 weeks, after about 8 weeks following encapsulation of one or more microparticles. Recordings can be conducted after about 9 weeks, 10 weeks, 12 weeks, 13 weeks, 15 weeks, 16 weeks, 18 weeks, 6 months, 7 months, 8 months, 9 months, 10 months, 11 months, 12 months, 16 months, 18 months, 20 months, 24 months, or more and any length of time therebetween following encapsulation of one or more microparticles.

As with any assay useful in the present disclosure, qualitative and quantitative readouts of a neuroid or cells of a neuroid may include baseline measurements in the absence of candidate agent; these baseline measurements are used in comparisons when the neuroid is contacted with a candidate agent.

Advantageously, neuroids of the present disclosure can be assayed at different time points along their maturation/development. Moreover, long-term effects of candidate agents or electrical stimulation on maturation, development, and/or function of a neuroid can be assessed over multiple time points.

Accordingly, an aspect of the present disclosure is an agent for producing a phenotypic change in a neuroid or a cell thereof. The agent is identified by a herein-disclosed method, e.g., screening assay.

### Microparticles

Neuroids of the present disclosure each comprises one or more internalized microparticles.

In embodiments, the one or more internalized microparticles provides an at least 10% increase in the expression of a neural marker, e.g., a cortical marker, by cells of the one or more neuroids, with the increase being relative to a neuroid prepared in an identical manner and with identical cells but that does not comprise the one or more internalized microparticles. The cortical marker may be FOXG1.

In embodiments, the one or more internalized microparticles provides an at least 20% increase in size of the one or more neuroids, with the increase being relative to a neuroid that is prepared in an identical manner and with identical cell types but that does not comprise the one or more internalized microparticles.

In embodiments, the one or more internalized microparticles provides an at least 20% increase in volume of the one or more neuroids, with the increase being relative to a neuroid that is prepared in an identical manner and with identical cell types but that does not comprise the one or more internalized microparticles.

In embodiments, the one or more internalized microparticles in a neuroid comprises a polymer, glass, hydrogel, plastic, silica, ceramic, or magnetic substance.

Examples of polymers include poly(hydroxyethyl methacrylate) (PHEMA), poly(ethylene terephthalate) (PET), poly(tetrafluoroethylene) (PTFE), fluorinated ethylene (FEP), poly(dimethyl siloxane) (PDMS) and other silicone rubber surfaces. Other examples of plastics or polymers, include dendritic polymers, such as poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), poly(ethylene-co-acrylic acid), or derivatives thereof. In embodiments, the one or more internalized microparticles in a neuroid comprises a polymer comprising polystyrene (PS).

Microparticles may comprise glass, quartz, silicon, metal or polystyrene. In some cases, the microparticles may comprise transparent glass, ceramic, metal, quartz, and polystyrene to enable transparent microscopic observation of culture cells. In some cases, the microparticle is nearly or fully optically transparent. The microparticle may have a refractive index that is substantial match with an aqueous medium (e.g., culturing medium) and cellular material, thereby, facilitating microscopic imaging. In some cases, the microparticles may be composed of glycophase glass (glycerol propylsilane bonded glass). Glass materials include soda-lime glass, pyrex glass, vycor glass, and quartz glass. In embodiments, the one or more internalized microparticles in a neuroid comprises glass. In embodiments, the one or more internalized microparticles in a neuroid comprises silica, e.g., mesoporous silica.

Microparticles may comprise a hydrogel. The hydrogel may be a water-swollen, cross-linked polymeric structure containing (1) covalent bonds produced by the reaction of one or more comonomers, (2) physical cross-links due to chain entanglements, (3) association bonds including hydrogen bonds or strong van der Waals interactions between chains, or (4) crystallites bringing together two or more macromolecular chains.

The hydrogel may comprise polymers, e.g., hydrophilic polymers that are linear or branched. In some cases, the polymers are synthetic. In some cases, the polymers are poly(ethylene glycol) molecules, wherein the poly(ethylene glycol) molecules are selected from the group comprising: poly(ethylene glycol), polyaliphatic polyurethanes, polyether polyurethanes, polyester polyurethanes, polyethylene copolymers, polyamides, polyvinyl alcohols, poly(ethylene oxide), polypropylene oxide, polyethylene glycol, polypropylene glycol, polytetramethylene oxide, polyvinyl pyrrolidone, polyacrylamide, poly(hydroxy ethyl acrylate), poly(hydroxyethyl methacrylate), and mixtures thereof.

The hydrogel may comprise naturally-derived biomaterials such as polysaccharides, gelatinous proteins, or ECM components comprising the following or functional variants thereof: agarose, alginate, chitosan, dextran, gelatin, laminins, collagens, hyaluronan, fibrin, amylose, amylopectin, carrageenan, and mixtures thereof, or are selected from the group of natural or synthetic complex tissue-derived matrices. Alternatively, the hydrogel may be formed of MATRIGEL, MYOGEL or CARTIGEL, or a combination of MATRIGEL, MYOGEL and CARTIGEL and a naturally derived biomaterial or biomaterials. In embodiments, the one or more internalized microparticles in a neuroid comprises a hydrogel comprising agarose.

In embodiments, the one or more internalized microparticles in a neuroid comprises a magnetic substance. In some cases, the magnetic substance is a metal such as a transition element selected from nickel, cobalt, iridium, iron, platinum, gold, silver, manganese, chromium, palladium, yttrium, neodymium, samarium, gadolinium, and terbium. The microparticles comprising magnetic substances may contain additional (non-magnetic) elements including copper, zinc, magnesium, rhenium, bismuth, and silicon and/or they may comprise iron-copper alloys, iron-platinum alloys, nickel, nickel-iron alloys, cobalt, cobalt-iron alloys, manganese, manganese-iron alloys, titanium, titanium-iron alloys, vanadium, vanadium-copper alloys, and magnetite (Fe3O4). The microparticles comprising magnetic substances may have physical properties other than magnetism, for example physical properties of semiconductors, may have a fixed magnetic pole (a permanent magnet), or may have a shifting magnetic pole.

A neuroid with internalized microparticles comprising a magnetic substance can be manipulated in a spatial location using an external magnetic field. An aspect of the present disclosure is a method of manipulating one or more neuroids in a culture vessel. The method comprising (a) providing one or more neuroids comprising at least one internalized microparticle comprising a magnetic substance; and (b) applying an external magnetic field to the one or more neuroids, thereby manipulating the one or more neuroids in the culture vessel.

In embodiments, manipulating neuroids in a culture vessel comprises adjusting the position and/or orientation of the neuroids by the applied external magnetic field, restricting motion of the one or more neuroids in their location in the culture vessel, and/or separating the one or more neuroids from a surface of the culture vessel. In embodiments, the external magnetic field is provided by a magnet that does not contact a liquid medium in the culture vessel.

In some examples, a microparticle additionally comprises poly L-lysine, geltrex, gelatin, mitogen, fibronectin, collagen I, collagen IV, fibrinogen, gelatin, glycosaminoglycans, gelatin methacrylate, fibrin, silk, pegylated gels, collagen methacrylate, basement membrane proteins, or any other biomaterial, or a combination thereof. The microparticle may comprise gelatin/chondroitin-6-sulfate/hyaluronan, methacrylate modified chondroitin sulfate, aldehyde-modified chondroitin sulfate, dermatan sulfate, poly (L-lactide)-g-chondroitin sulfate, PEG, EDC cross-linked chondroitin sulfate, thiolated chondroitin sulfate, heparin, pluronic F127 nanogel, or any other polymer comprising a functional modification to chondroitin sulfate, or a combination thereof. The microparticle may comprise a co-polymer; the copolymers may be graft copolymers comprising a polyacetal backbone with pendant poly-ethylene glycol side chains, linear poly (N-isopropylacrylamide-co-butylmetacrylate-co-acrylic acid) terpolymers, and poly (N-isopropylacrylamide-co-methacrylic acid).

The microparticle may vary in stiffness. In some cases, the stiffness of microparticle may control the growth and morphogenesis of a neuroid. One type of neuroid (e.g., comprising or derived from a first cell type) may prefer microparticles having higher stiffness than a second type of neuroid (e.g., comprising or derived from a second cell type). For example, a cortical neuroid may require a stiffer microparticle than a retinal neuroid. Some hydrogel-based microparticles may form a viscous solution or semi-solid media, or may form a gel with a stiffness (shear or elastic modulus) between about 50 Pa and about 50 kPa.

The microparticles that are internalized into a neuroid can have a variety of shapes. In general, the microparticles provided herein are spherical or spheroid, cubic or near-cubic, ovoid, rhomboid, or flattened disks. The microparticle can have at least one face that is a polygon (e.g., triangle, rectangle, square, quadrilateral, trapezoid, and parallelogram). In some cases, at least two faces of the microparticle have different shapes; for example, one face may be square while another face is a flat triangle. The faces of the microparticle may be curved, flat, or irregular, or some combination thereof. In some cases, one face is curved, while the others are flat. In some cases, all faces of the microparticle are flat. In some cases, all faces of the microparticle are curved. In some cases, the microparticle may have one or more asymmetries or imperfections, e.g., rough-cut irregular microparticles or randomly shaped. In embodiments, a microparticle may comprise a three-dimensional scaffold structure. Such scaffolds may include a variety of components which may have differing shapes and/or sizes. Examples of such scaffold include those manufactured by Prellis Biologics.

A microparticle may be solid or porous. In some cases, the microparticle can have a porosity sufficient to allow passage of molecules having a molecular weight greater than about 10,000 Daltons; in some cases, the microparticle has a porosity sufficient to admit proteins and other biological macromolecules of a molecular weight up to and greater than 10,000 Daltons, such as from 10,000 to 1,000,000 Daltons. A pore, as used herein, can have a regular or irregular morphology. It should be recognized that not all pores may be of the same diameter.

Microparticles internalized into a neuroid may be heterogenous and comprise different sized and shaped microparticles. In some cases, the sizes and shapes are random. In some cases, the microparticles are homogenous in size and shape.

As used herein, the term microparticle here can include an oblong arrangement with an aspect ratio of a prolate dimension to a perpendicular dimension less than 2:1. In some cases, the term microparticle as used herein does not include an oblong arrangement with an aspect ratio of a prolate dimension to a perpendicular dimension of at least 2:1, at least 3:1, at least 4:1, at least 5:1 or at least 10:1. Thus, as used herein, the term microparticle does not include fibers, filaments, or rods, unless such rod, filament or fiber has an aspect ratio of length:width (broadest width) that is less than 2:1.

Microparticles that are internalized into a neuroid can have a variety of sizes, with a height, width, length, or diameter of the microparticle being from about 0.1 μm to about 1 mm. Thus, a microparticle have a height, width, length, or diameter of no more than 1 mm and at least 0.01 μm, 0.05 μm, 0.1 μm, 0.2 μm, 0.4 μm, 0.5 μm, 1 μm, 2 μm, 3 μm, 5 μm, 7 μm, 8 μm, 10 μm, 20 μm, 30 μm, 40 μm, 50 μm, 60 μm, 70 um, 80 um, 90 um, 100 um, 200 um, 300 um, 400 um, 500 um, 600 μm, 700 μm, 800 μm, 900 μm, and 100 μm. In embodiments, the one or more internalized microparticles is less than 500 μm in diameter and/or the one or more internalized microparticles is greater than 2 μm in diameter.

In some cases, the microparticle is degradable. In other cases, the microparticle is non-degradable. The microparticles may be removed from neuroids via mechanical agitation or by breakdown/degradation of the microparticle. The rate of degradation can be controlled by hydrolysis, abiotic degradation processes or cleavage by extracellular enzymes. Degradation of microparticles comprising a hydrogel may be induced by application of a chemical, light energy, and/or a digestive enzyme.

In some cases, a microparticle is pretreated to improve the microparticle's adhesivity to a cell. The pretreatment may comprise use of bovine serum albumin, collagen, alginic acid gel, poly vinyl alcohol, or polyacrylamide. The microparticle-cell interactions may be stabilized by negatively charging the microparticle's surface, e.g., by coating it with poly-L-lysine. The microparticle may be treated with hexamethyldisilazane followed by light exposure. The microparticle's surface may undergo chemical modification to facilitate adsorption and attachment of functional agents.

In some examples, the microparticle possesses the ability to self-assemble upon temperature variation. The self-assembly of a microparticle may be induced by temperature-controlled intermolecular hydrogel bonds. In some cases, materials that respond to temperature can act as a driving stimulus to trigger protein release. pNIPAM (a polymer of the subunit N-isopropylacrylamide) is an example of a temperature responsive material; it has a lower critical solution temperature (LCST) of 32° C. Other external triggers such as pH and ion concentration can be used to induce assembly of microparticles.

In some cases, the mesh size of the hydrogel material of the microparticle can be affected enzymatically or by pH. Chitosan-based pH- and glucose-responsive microparticle can be fabricated by an electrospraying technique. Microparticles containing t-butyl methacrylate and 2-(t-butylamino)ethyl methacrylate synthesized with emulsion polymerization and mixed with the solution of methacrylic acid and N-vinyl pyrrolidone have variable mesh size regulated by pH.

In some cases, the functionalized microparticle can function as an organizer or scaffold for the developing neuroid. The mechanical properties of a microparticle scaffold can be adjusted depending on the desired neuroid type. Here, porosity, stiffness, and yield limit, as examples can be adjusted as needed. In some cases, the microparticles provides structural support for the neuroid.

In some cases, neuroid patterning can be stabilized by microparticle-microparticle interactions. Here, microparticles assemble into networks using cross-linking. The mechanical properties of a neuroid may be enhanced by cross-linking of microparticles. In some cases, microparticles comprising or attached with biotin self-assemble through biotin-streptavidin crosslinking; here, biotin functionalized microparticles are mixed with cells and avidin to form stable assemblies. In some cases, microparticles are doubly crosslinked to covalently connect particles using connecting polymer linkers for tunable viscoelastic properties; for example, internally crosslinked HA microparticles can be modified with surface aldehyde functionalities that can be additionally cross-linked through soluble HA modified with adipic dihydrazide.

The microparticle may be surface-modified. The surface of the microparticle can be activated via modification of a nucleophile, such as an amine or hydroxyl, followed by coupling to a functional agent, e.g., a peptide, via another nucleophile such as an amine or hydroxyl or thiol. By way of example, the activation of surface hydroxyl groups may be accomplished through treatment with agents such as tresyl chloride, glutaraldehyde, cyanuric chloride, sulfonyl chlorides, cyanogen bromide; surface hydroxyls may be added via benzoin with potassium tert-butoxide in dimethyl sulfoxide. Treatment with these particular surface activators is followed by a procedure by which a functional agent is covalently linked to the hydroxyl group. Additionally, active carboxyl groups may be introduced on the surface by using, for example, succinic anhydride.

In embodiments, polydopamine (PDA) or Protein A serves as a linker between a microparticle and functional agent. PDA or Protein A help attachment/coupling of a protein-based functional agent to the microparticle.

A neuroid typically comprises a plurality of microparticles. However, in embodiments, a neuroid comprises a total volume of internalized microparticles that is less than one third of a total volume of the neuroid.

### Functional Agents

In some cases, the microparticle comprises a functional agent. In some cases, functional agents can be tethered to the microparticle surface. In some cases, functional agents can be diffusible from the microparticle. In some cases, the functional agents can be release in a controlled manner from the microparticles. In some cases, the functional agents can be released in a time-delayed profile from the microparticle. In embodiments, the microparticles are coated with or coupled to one or more functional agents. In embodiments, the microparticles encapsulates one or more functional agents.

Useful functional agents affect one or more of cell adhesion, spatial control of cell adhesion, cell growth, cell proliferation, cell organization, cell differentiation, cell repair, cell activity, cell maturation, and/or cell regeneration. The functional agent may be selected from the group consisting of: growth factors, growth factor receptors, transcriptional activators, translational promoters, anti-proliferative agents, growth hormones, anti-rejection drugs, anti-thrombotic agents, anti-coagulants, stem cell or gene therapy agents, antioxidants, free radical scavengers, nutrients, enzymes, co-enzymes, ligands, cell adhesion peptides, peptides, proteins, nucleic acids, DNA, RNA, polysaccharides, sugars, nutrients, hormones, neurotransmitters, antibodies, immunomodulating agents, growth factor inhibitors, growth factor receptor antagonists, small molecules, chemical compounds and biologics. In some examples, the functional agent is a moiety disposed on a surface of the microparticle and is selected from the group consisting of: a protein, a peptide, a polysaccharide, a sugar, a toxin, an antibody, an aptamer, and a combination thereof.

Functional agents may be used individually or in a mixture of two or more types; as examples, a microparticle can comprise 1, 2, 3, 4, 5, 6, 7, 8, 9, 10, 11, 12, 13, 14, 15, 16, 17, 18, 19 or 20 types of functional agents.

In some cases, a plurality of functional agents can be layered to form continuous gradients of functional agents for patterning of neuroids.

In some cases, microparticles comprise extracellular matrix molecules as a functional agent. Examples include, but are not limited to, fibronectin, laminin, collagens, and proteoglycans. Other growth effector molecules useful for tethering include cytokines, such as the interleukins and GM-colony stimulating factor, and hormones, such as insulin. In some embodiments, a functional agent comprises a chemical substance that facilitates adhesion and proliferation of cells, examples of which include integrin, laminin, hemidesmosome, selectin, CDH1, ICAM-1, CDH2, CD31, CD29, L1, L-selectin, PTK2, carcinoembryonic antigen, VLA-4, CD44, Vinculin, Cadherin, Integrin-beta 3, vitronectin, P-selectin, CAM-1, Desmocollin, ICAM3, Plakoglobin, P-selectin, Talin, CD24, desmoglein-1, neurexin, and CD58. A functional agent may comprise glycoproteins that promote attachment and growth of human/animal cells, with examples including fibronectin, vitronectin, collagen, laminin and thrombospondin.

In embodiments, a microparticle is coupled to one or more functional agents selected from fibronectin, laminin, collagen IV, and MATRIGEL.

In embodiments, a microparticle comprises polydopamine (PDA) or Protein A which serves as a linker between the microparticle and functional agent, e.g., a protein-based functional agent to the microparticle.

A microparticle may comprise a therapeutic or diagnostic agent as a functional agent. Examples of therapeutic agents include proteins, such as hormones, antigens, and growth effector molecules; nucleic acids, such as antisense molecules; and small organic or inorganic molecules such as antibiotics, steroids, decongestants, neuroactive agents (e.g., a neurotransmitter such as dopamine), anesthetics, and sedatives. Examples of diagnostic agents include radioactive isotopes, radiopaque agents and magnetic compounds.

A microparticle may comprise a growth effector molecule that interacts with cell surface receptors and regulate the adhesion, growth, replication, or differentiation of target cells or tissue as a functional agent. Examples of growth effector molecules are growth factors and extracellular matrix molecules. Examples of growth factors include epidermal growth factor (EGF), platelet-derived growth factor (PDGF), transforming growth factors (TGF-α, TGF-β, hepatocyte growth factor, heparin binding factor, insulin-like growth factor I or II, fibroblast growth factor, erythropoietin, nerve growth factor, bone morphogenic proteins, muscle morphogenic proteins.

In some cases, a microparticle comprises a functional agent that boosts nutrient transport and/or vascular differentiation of a neuroid. For example, incorporated microparticles at the center of the neuroid can act as synthetic cells and can help to mimic dense-packed tissue without consuming nutrients, and/or oxygen, which may be in low supply at the core of a neuroid. In some cases, the microparticle promotes higher local oxygen concentrations by combining a polyvinylpyrrolidone/hydrogen peroxide core with a catalase to generate higher local oxygen generation in the neuroid. In some cases, the incorporated microparticles can comprise agents that improve nutrient transport into and out of the neuroid. In some cases, the agents are PIGF, VEGF or bGFG. The microparticle may comprise a functional agent that locally adjusts pH. In some cases, the microparticle helps deliver nutrients within a neuroid by enabling the formation of vasculature. The incorporated microparticle may provide local, controlled release of diffusing proteins or small molecules within the neuroid.

In embodiments, a neuroid further comprises at least one additional microparticle comprising one or more functional agents selected from the group consisting of: a polypeptide, a polysaccharide, a small molecule, a nucleic acid, an imaging agent, MATRIGEL, hydrogel, and a sensor, or any combination thereof.

In embodiments, a microparticle comprises a polymer comprising polystyrene (PS). The PS-comprising microparticles may be coated with polydopamine (PDA) or Protein A, which serves as a linker between the PS-comprising microparticles and a functional agent. In embodiments, the PS-comprising polystyrene microparticle may be coupled to one or more herein-disclosed functional agents. The functional agents are selected from fibronectin, laminin, collagen IV, and MATRIGEL. In embodiments, the functional agent is fibronectin. Alternately, the PS-comprising microparticle may lack a functional agent.

In embodiments, a microparticles comprises a hydrogel comprising agarose. The agarose microparticle may be coated with polydopamine (PDA) or protein A which serves as a linker between the agarose-comprising microparticle and a functional agent. In embodiments, the agarose-comprising microparticle may be coupled to one or more herein-disclosed functional agents. The functional agents may be selected from fibronectin, laminin, collagen IV, and MATRIGEL. Alternately, the agarose-comprising may lack a functional agent. In this embodiment, neuroid further comprises at least one additional microparticle comprising MATRIGEL as a functional agent.

In embodiments, a microparticle comprises a magnetic substance. In embodiments, the microparticle comprising a magnetic substance may be coated with protein A or PDA, which serves as a linker between the microparticle comprising a magnetic substance and a functional agent. In embodiments, the microparticle comprising a magnetic substance may be coupled to one or more herein-disclosed functional agents. The functional agents may be selected from fibronectin, laminin, collagen IV, and MATRIGEL. Alternately, the microparticle comprising a magnetic substance may lack a functional agent.

In embodiments, the microparticle comprises glass. The glass-comprising microparticle may be coated with protein A or PDA, which serves as a linker between the glass-comprising microparticle and a functional agent. In embodiments, the glass-comprising microparticle may be coupled to one or more herein-disclosed functional agents. The functional agents may be selected from fibronectin, laminin, collagen IV, and MATRIGEL. Alternately, the glass microparticle may lack a functional agent.

In embodiments, the microparticle comprises silica, e.g., mesoporous silica. The mesoporous silica-comprising microparticle may be coated with protein A or PDA, which serves as a linker between the mesoporous silica-comprising microparticle and a functional agent. In embodiments, the mesoporous silica-comprising microparticle may be coupled to one or more herein-disclosed functional agents. The functional agents may be selected from fibronectin, laminin, collagen IV, and MATRIGEL. Alternately, the mesoporous silica microparticle may lack a functional agent.

Another herein-disclosed functional agent may be a peptide for cell-surface receptor recognition sequences, for example, RGD, YIGSR, or REDV. Minimal cell surface receptor recognition sequences may include the GRGD (Gly-Arg-Gly-Asp), GYIGSRY (Gly-Tyr-Ile-Gly-Ser-Arg-Tyr), GYIGSR (Gly-Tyr-Ile-Gly-Ser-Arg), GRGDY (Gly-Arg-Gly-Asp-Tyr), YIGSR (Tyr-Ile-Gly-Ser-Arg), RGD (Gly-Arg-Asp), REDV (Arg-Glu-Asp-Val), GREDV (Gly-Arg-Glu-Asp,Val), GREDVY (Gly-Arg-Glu-Asp-Val-Tyr), RGDS (Arg-Gly-Asp-Ser), GRGDS (Gly-Arg-Gly-Asp-Ser), RGDF (Arg-Gly-Asp-Phe), GRGDF (Gly-Arg-Gly-Asp-Phe), PDSGR (Pro-Asp-Ser-Gly-Arg), GPDSGR (Gly-Pro-Asp-Ser-Gly-Arg), GPDSGRY (Gly-Pro-Asp-Ser-Gly-Arg-Tyr), IKVAVC (Ile-Lys-Val-Ala-Val-Cys), GIKVAV (Gly-Ile-Lys-Val-Ala-Val), IKVAVY (Ile-Lys-Val-Ala-Val-Tyr), GIKVAVY (Gly-Ile-Lys-Val-Ala-Val-Tyr) amino acid sequences. Such fragments can contain either the cell attachment sequence of many surface adhesion molecules (RGD) or one of the cell attachment sequences of laminin (YIGSR and PDSGR), a particular surface adhesion protein, or the cell adhesion molecule fibronectin (REDV). The IKVAV peptide from laminin can also be useful for particular cells. In some cases, the peptides may further include a C-terminal Y for radioiodination. The N-terminal G can be used as a spacer with the particular peptide between the adhesive peptide and the surface. The small peptides can be used to provide cell receptor recognition sites required for cell adhesion on the treated surface.

In some cases, proteins can be adsorbed onto microparticles. For example, the core of the microparticle may comprise polystyrene and negatively charged shell may comprise crosslinked NIPAM, methylenebisacrylamide, and acrylic acid. As ion concentration is increased, binding affinity of the lysozme to the microgel decreases.

Protein functional agents may be naturally derived or recombinant.

In some cases, the herein-disclosed functional agent is a fluorescent moiety.

A herein-disclosed functional agent may comprise a sensor that can sense the microenvironment (or microclimate) of neuroid. Illustrative sensors can detect and relay temperature, pH level, oxygen levels, carbon dioxide levels, nutrient-content, osmolarity, electrical charge, and the presence of pharmaceutical agents. The sensor may be a peptide that is sensitive to protease activity. For example, microparticles can comprise a peptide-based FRET sensor that reports local proteolytic activity of collagenase through changes in fluorescence.

In embodiments, a functional agent is reversibly attached to the microparticle. In this embodiment, the functional agent releases from and diffuses away from the microparticle over time; thereby, forming a concentration gradient, within the neuroid, for the functional agent, with the gradient originating at an internalized microparticle.

Hydrogel microparticles more readily release their functional agents in a diffusion-based manner. Parameters such as mesh size can be adjust to control release profiles, with a smaller mesh size hindering diffusion-based release of the agent. Hydrogel microparticle mesh sizes may range from 5 to 100 nm. Additional control over diffusion-based release can be achieved by incorporating molecules such as heparin to take advantage of affinity interactions with the functional agent. Degradation of the microparticle over time can contribute to the release of the functional agent, especially if the functional agent naturally has low diffusivity. Polymer weight percentage, molecular weight and crosslinking density, can also be adjusted to control degradation-based functional agent release. Microparticles with enzymatically-degradable linkages will have a release profile dependent on the cellular microenvironment (i.e., the presence of the enzyme capable of degrading the links). External stimuli (temperature, pH, and ion concentration) can also be adjusted to functional agent release.

In some cases, a functional agent is coupled to a microparticle by co-incubation or co-agitation. Microparticles can be cross-linked to functional agents using methods such as ultraviolet (UV) irradiation, electrospraying, dehydrothermal treatment and N-(3-Dimethylaminopropyl)-N′-ethylcarbodiimide hydrochloride (EDC) treatment.

### Methods for Producing Neuroids Comprising Microparticles

An aspect of the present disclosure is a method of producing a neuroid comprising at least one internalized microparticle. The method comprises (a) providing a plurality of cells in a culture vessel including at least one cell is a stem cell, e.g., an induced pluripotent stem cell (iPSC); (b) contacting the plurality of cells in the culture vessel with one or more microparticles; and (c) culturing the plurality of cells with the one or more microparticles, thereby producing a neuroid comprising at least one internalized microparticle.

In embodiments, the one or more microparticles comprises a polymer, glass, hydrogel, plastic, silica, ceramic, or magnetic substance, as described elsewhere herein.

In embodiments, the one or more microparticles comprises at least one functional agent, as described elsewhere herein. In embodiments, the one or more microparticles are coated with or coupled to one or more functional agents selected from fibronectin, laminin, collagen IV, and MATRIGEL. The coating may be by co-incubation and/or the coupling may be by chemical cross-linking, co-incubation, co-agitation, or centrifugation.

The plurality of cells forming the neuroid may be contacted with one or more microparticles. In some cases, the plurality of cells forming the neuroid are contacted with at least 1, 3, 6, 10, 14, 16, 19, 25, 30, 32, 35, 38, 40, 42, 45, 48, 50 55, 60, 65, 70, 75, 80, 85, 90, 100, 110, 120, 140 , 160, 180, 200, 225, 250, 280, 300, 350, 330, 350, 380, 400, 420, 450, 480, 500 microparticles. In some cases, the plurality of cells forming the neuroid can contain at least 1, 3, 6, 10, 14, 16, 19, 25, 30, 32, 35, 38, 40, 42, 45, 48, 50 55, 60, 65, 70, 75, 80, 85, 90, 100, 110, 120, 140 , 160, 180, 200, 225, 250, 280, 300, 350, 330, 350, 380, 400, 420, 450, 480, 500 microparticles.

In embodiments, the plurality of cells is provided to a cell culture vessel comprising the one or more microparticles or the one or more microparticles is added to a cell culture vessel comprising the plurality of cells; the plurality of cells may have formed a cell aggregate before adding the one or more microparticles.

In some cases, the plurality of cells forming the neuroid are added in the form of a suspension of single cells. In some cases, microparticles are added to the plurality of cells or cells are added to the microparticles. In some cases, the plurality of cells may already be in the form of a cell aggregate e.g., embryoid body, when the microparticles are added. In some cases, after microparticles are added, the plurality of cells aggregate to form an embryoid body comprising internalized microparticle. In some cases, the plurality of cells forming the neuroid are added on top of the surface of the cell culture vessel to which the microparticles are already added.

In embodiments, the plurality of cells is co-incubated with the one or more microparticles for at least 12 hours; culturing the plurality of cells with the one or more microparticles occurs for up to 8 hours, up to 16 hours, up to 24 hours, up to 2 days, up to 3 days, up to 7 days, or up to 10 days. In some cases, after incubation for a period of time, e.g., 24 hours, the cells are aggregated at the bottom of the cavities. In some cases, the aggregated cells form an embryoid body. In some cases, one or more microparticles is internalized by the embryoid body.

In embodiments, the neuroid comprising at least one internalized microparticle for at least 3 days, at least 7 days, at least 10 days, at least 14 days, at least 20 days, at least 30 days, at least 90 days, at least 200 days, at least 350 days or at least 500 days.

A culture vessel for growing a neuroid contains a suitable a cell culture medium.

In some examples, the cell culture medium may comprise cell-protecting additives that can affect the surface shear, dilatational viscosities, dynamic surface tension, foaminess and foam stability and other interfacial properties that can contribute to cell protection against shear damage. Examples of such additives, include, but are not limited to, Methocel, Pluronic F68, or polyvinyl alcohol (PVA), polyethylene glycol (PEG) or polyvinyl-pyrrolidone (PVP), serum (FBS), and serum albumin (BSA).

In embodiments, the method further comprises overlaying the plurality of cells and the one or more microparticles with a biocompatible matrix and/or further comprises aggregating the plurality of cells and the one or more microparticles by co-incubation, centrifugation, or co-agitation. The biocompatible matrix, e.g., a three-dimensional matrix, may be a hydrogel, e.g., comprising polyvinyl alcohol, sodium polyacrylate, acrylate polymers and copolymers with an abundance of hydrophilic groups.

In some cases, the biocompatible matrix is a hydrogel. The hydrogel of the overlay may be dilute such that it forms a viscous solution or semi-solid media, or may form a gel with a stiffness (shear or elastic modulus) between about 50 Pa and about 50 kPa. In some cases, the biocompatible material overlaying the multicellular aggregate is of a similar or identical composition to the material of the microparticle.

A biocompatible matrix may comprise components to guide the growth and development of the neuroids. For example, the biocompatible matrix may comprise collagen type 1. In embodiments, the biocompatible matrix can comprise extracellular matrix from the Engelbreth-Holm-Swarm tumor or any component thereof such as laminin, collagen, preferably type 4 collagen, entactin, and optionally further heparan-sulfated proteoglycan or any combination thereof, e.g., MATRIGEL. In embodiments, the biocompatible matrix comprises a concentration of at least 3.7 mg/ml containing in parts by weight about 60-85% laminin, 5-30% collagen IV, optionally 1-10% nidogen, optionally 1-10% heparan sulfate proteoglycan and 1-10% entactin. MATRIGEL'S solid components usually comprise approximately 60% laminin, 30% collagen IV, and 8% entactin. Entactin is a bridging molecule that interacts with laminin and collagen. The biocompatible matrix may further comprise growth factors, such as any one of EGF (epidermal growth factor), FGF (fibroblast growth factor), NGF, PDGF, IGF (insulin-like growth factor), especially IGF-1, TGF-β, tissue plasminogen activator. The biocompatible matrix may lack any of these growth factors. In some cases, the biocompatible matrix includes collagen, gelatin, chitosan, hyaluronan, methylcellulose, laminin and/or alginate. The biocompatible matrix may be a gel, in particular a hydrogel.

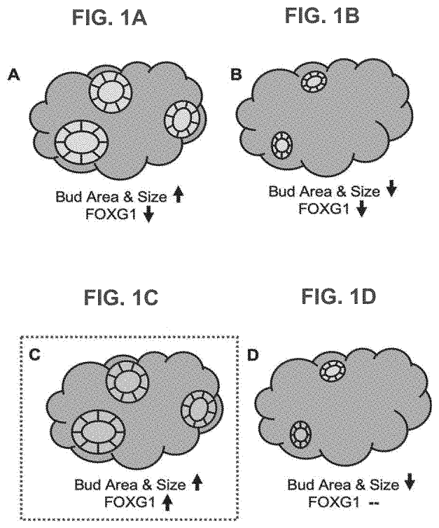

In some cases, the neuroids form buds/rosettes (e.g., neuronal rosettes). In some neuroids, a rosette is an indication of native cortical architecture. In embodiments, a cortical neuroid comprises large and numerous cortical rosettes with a forebrain identity indicated by the marker FOXG1, as illustrated in FIG. 1C.

The distribution of the microparticles in a culture vessel can guide the development of the neuroid. In some cases, the microparticles can be homogenously distributed within the neuroid or can be distributed non-uniformly throughout the neuroid. In the latter cases, the microparticles can be restricted to a single region within the neuroid. In some cases, the microparticles are incorporated into different regions of the neuroid to better mimic the complex organization of native tissues.

In some cases, neuroids are generated using free-floating culture methods.

Neuroids can be cultured in culture vessels having low-adhesion or non-adhesive surfaces. Such a low-adhesion surface, e.g., an ultra-low adhesion plate, helps prevent binding of cells to the vessel surface.

In some cases, the surface of a culture vessel is a substantially flat, two-dimensional surface, i.e., without topological features.

In some cases, the surface of the culture vessel has topological features of various sizes, shapes and depths, e.g., serrations, cavities, or microwells. In embodiments, a surface of the culture vessel comprises one or more cavities or microwells which provide consistent three-dimensional shape and size among neuroids. In these cases, a resulting neuroid will comprise a shape and size that corresponds to the cavity or microwell. An advantage of this method is the production of a plurality of similar sized and shaped neuroids, and, likely, cell density. This uniformity improves accuracy and reproducibility of subsequent assays, as variability due to neuroid size, shape, and cell density is reduced.

In some cases, the depth of a cavity or microwell is great enough that every cell to form the neuroid in contained in a cavity or microwell. In some embodiments, the neuroid is growth restricted in one or more dimensions. For example, when added cells completely cover the bottom of a cavity or microwell, the neuroid is restricted in growth in the vertical direction.

In some cases, the plurality of cells forming the neuroid and microparticles can be aggregated by forced spatial confinement, such as centrifugation of cells and microparticles. In some cases, the plurality of cells forming the neuroid and the microparticles are aggregated by seeding cells in a low-adhesion culture plates, e.g., which helps prevent binding of cells to the culture plate's surface. In some cases, the plurality of cells forming the neuroid and the microparticles are aggregated by seeding cell-microparticle suspensions in swellable medium. e.g., methylcellulose or dextran. In some cases, cells and functionalized microparticles self-associate through non-specific interactions and merely by co-agitation for a given period of time.

In some embodiments, patterning of a neuroid is influenced by the intensity of cohesion among the cells of the neuroid itself or the cohesivity between the neuroid and the biocompatible matrix. In some cases, the cohesivity of the cells within the neuroid may range from 0.2 dyne/cm to 20 dyne/cm. In some examples, the interfacial tension of the surface of the neuroid in contact with the biocompatible matrix can range from 0.5 mN/m to 500 mN/m (as measured by tensiometry combined with the solution of the Laplace equation).

In embodiments, culturing the neuroid comprises positioning the neuroid at an air-liquid interface. Here, the neuroid is not submerged in liquid on at least one surface, e.g., the upper surface that is opposite the culture vessel surface or biocompatible matrix upper surface.

A neuroid may be transferred to a surface of a biocompatible matrix or to an elevated topological feature (e.g., a pedestal) of a culture vessel and placed at an air-liquid interface; there, culturing continues. Culturing a neuroid at an air-liquid interface induces formation of a compacted neuroid. The neuroid may be elevated by a height ranging from 1 mm to 10 cm from the bottom surface of the cell culture medium. In some cases, the neuroid is placed at an air-liquid interface by adjusting the cell culture media level to be below the top surface of the topological feature. In some cases, the liquid culture medium is in contact with one or more surfaces of the neuroid, but does not submerge the neuroid.

In embodiments, culturing a neuroid at an air-liquid interface allows for generation of a neuroid having lower necrosis levels compared to a substantially similar neuroid that is not cultured at an air-liquid interface. In some cases, necrosis levels are reduced by at least 5%, 7%, 10%, 14%, 18%, 20%, 25%, 30%, 40%, 45%, 50%, 60%, 70%, 80%, 90% or 99%.

A method of generating a neuroid at the air-liquid interface may comprise steps of: (a) providing a pedestal comprising a plurality of cells comprising at least one stem cell or a multicellular aggregation of cells comprising at least one stem cell, (b) contacting the plurality of cells or multicellular aggregation of cells with a microparticle comprising a functional agent, thereby allowing internalization of the microparticle, (c) culturing said cell aggregation comprising the internalized microparticle in a three dimensional matrix, wherein said cells are allowed to differentiate, thereby expanding said cells, and (d) culturing said expanded cells of step (c) in a neuroid culture matrix at an air-liquid interface to generate a compacted neuroid.

In embodiments, a neuroid is grown at the air-liquid interface for varying lengths of time, e.g., for 1 day, 2 days, 3, days, 4 days, 5 days, 1 week, 2 weeks, 3 weeks, 4 weeks, 5 weeks, weeks, 8 weeks, 9 weeks, 10 weeks, 12 weeks, 13 weeks, 15 weeks, 16 weeks, 18 weeks, 6 months, 7 months, 8 months, 9 months, 10 months, 11 months, 12 months, 16 months, 18 months, 20 months, 24 months, or more and any length of time therebetween.

In some cases, the cell aggregates or compacting structures comprising the functionalized microparticle may be overlaid with a biocompatible matrix. In some cases, the biocompatible matrix is a dilute or non-dilute hydrogel. A pro-organ at the air-liquid interface (ALI) may be overlaid with a thin coating of hydrogel. In some cases, the hydrogel is overlaid on the neuroid, after the neuroid is placed on a pedestal. In some cases, the neuroid is placed on a pedestal and then overlaid with a hydrogel cover. In some cases, appropriate media, and the combinations of nutrients or signaling factors, such as growth factors and morphogens, are added to the biocompatible matrix.

In embodiments, a plurality of neuroids can be assembled to form a fusion neuroid which include two or more previously-distinct neuroids. As examples, a fusion neuroid comprises two neuroids each comprising or derived from cells of the same line; a fusion neuroid comprises two neuroids each comprising or derived from cells of a different line; and a fusion neuroid comprises three neuroids each comprising or derived from cells of different lines. Such fusion neuroids approximate junctions/connections between different regions of the mammalian nervous system. A fusion neuroid may retain at least one of its respective unique characteristics, e.g., spatial organization, molecular marker expression, and brain region representation.

In embodiments, neuroids in the set comprise a fusion and/or contact between a neuroid and a non-neuroid tissue, e.g., a native neural tissue, a blood vessel tissue, and glial tissue, including microglia.

Neuroid creating may comprise formation of embryoid bodies (EBs). An Illustrative embryoid body or neuroid production protocol may start with isolated embryonic stem cells or pluripotent stem cells (e.g., iPSC) or from cells that are derived from explants from human tissues.

Culture conditions for neuroid development can provide an environment permissive for differentiation, in which cells proliferate, differentiate, or mature in vitro. Such conditions may also be referred to as differentiative conditions. Features of the permissive environment include the medium in which the cells are cultured, any growth factors or differentiation-inducing factors that may be present, and a supporting structure (such as a substrate on a solid surface) if present. Differentiation may be initiated by formation of embryoid bodies (EB), or similar structures. For example, EB can result from overgrowth of a donor cell culture, or by culturing ES cells in suspension in culture vessels having a substrate with low adhesion properties.

The differentiation medium may comprise supplements including growth factors, antibiotics, vitamins metabolites, and hormones, synthetic or natural with similar properties. In one embodiment, the differentiation medium comprises Dulbecco's Modified Eagle medium: Nutrient Mixture F-12 (DMEM/F12) (ThermoFisher Scientific), B27 supplement, bFGF, and EGF. In an embodiment, the neuroid's culture medium includes DMEM, B27 supplement, bFGF, EGF, and hydrocortisone. Illustrative media for culturing embryoid bodies or neuroids can include Aggrewell™ medium (AW; StemCell Technologies, Inc). The media may contain supplements such as N2, heparin, glutamax, B27, 2-mercaptoethanol, Vitamin A, insulin, amino acids to enhance or support neural differentiation. Various biological agents or factors may be used in combination with the media, e.g., basic FGF, noggin, the small molecule TGF-beta inhibitor SB431542, Activin A, BMP-4, Wnt, epidermal growth factor (EGF), ascorbic acid, retinoic acid, bovine brain extract, heparin, hydrocortisone, gentamicin, fetal bovine serum, Insulin-like growth factor (IGF), and vascular endothelial growth factor (VEGF). In embodiments, endothelial cells may be encouraged to undergo proliferation and specification to an avascular phenotype. For example, endothelial cells can be cultured in brain microvascular endothelial cell (BMEC) medium including, e.g., endothelial serum-free medium supplemented with 20 ng/ml of FGF, 1% platelet-poor plasma-derived bovine serum, and 10 μM retinoic acid to form a brain microvascular phenotype.

During development of a neuroid, cell aggregates can exhibit a spatial organization patterns. For example, cells in the neuroid can be organized along a dorsal-ventral axis (roof, alar, basal and floor plate) and/or a rostral-caudal axis (telencephalon, diencephalon, mesencephalon and rhomboid encephalon and spinal cord). The neuroid can form polarized neuroepithelial structures and a neuroepithelial sheet. In some cases, the neuroid of the present disclosure comprises both self-renewing progenitors and differentiated cell types that show migratory motion. In some cases, the neuroid forms stratified cortical-like tissues containing 1, 2, 3, 4, 5, or 6 layers. In some cases, the neuroid recapitulate the spatial and temporal events leading to the formation of layered structures in the mammalian brain. In some cases, the neuroid adopts a rostral-hypothalamic fate. In some cases, the neuroid adopts a regional fate (e.g., olfactory bulb, rostral and caudal cortices, cortical hem, and choroid plexus). In some cases, the neuroid adopt an adenohypophysis fate. In some cases, neuroid comprises features of outer radial glial cells.

In embodiments, a method of the present disclosure further comprises isolating a differentiated neural cell of interest from the neuroid, e.g., the cell of interest isolated based on the presence or absence of a neural marker. The neural marker may be a cortical marker, e.g., FOXG1.

A variant of the above-described methods is a method of producing a neuroid comprising at least one internalized microparticle. The method comprises steps of (i) providing a plurality of cells in a culture vessel, wherein at least one cell is a stem cell; (ii) contacting the plurality of cells in the culture vessel with one or more microparticles lacking a functional agent; (iii) culturing the plurality of cells with the one or more microparticles lacking a functional agent; (iv) contacting the one or more microparticles with MATRIGEL, thereby allowing the microparticles to absorb the MATRIGEL; and (v) further culturing the plurality of cells with the one or more microparticles that have absorbed MATRIGEL to produce a neuroid comprising at least one internalized MATRIGEL-absorbed microparticle.

In embodiments, step (iii) occurs for up to 8 hours, up to 16 hours, up to 24 hours, up to 2 days, up to 3 days, up to 7 days, or up to 10 days and/or step (iv) occurs after completion of step (iii). In embodiments, the method further comprises culturing the neuroid comprising at least one internalized MATRIGEL-absorbed microparticle for at least 3 days, at least 7 days, at least 10 days, at least 14 days, at least 20 days, at least 30 days, at least 90 days, at least 200 days, at least 350 days or at least 500 days. The culturing of the plurality of cells with the one or more microparticles lacking a functional agent delays ingestion of the microparticles which provides improved rosette formation and area in the neuroid once contacted the one or more microparticles with MATRIGEL. The improved rosette formation and area is relative to a neuroid lacking a microparticle comprising MATRIGEL.

Another variant of the above-described methods is a method of producing a neuroid comprising at least one internalized microparticle. The method comprises steps of (I) providing a plurality of cells in a culture vessel, wherein at least one cell is a stem cell; (II) culturing the plurality of cells in the absence of a microparticle until a developing neuroid has formed or begun to form; (III) adding one or more microparticles comprising MATRIGEL and allowing the developing neuroid to internalize the microparticles comprising MATRIGEL; and (IV) further culturing the developing neuroid to produce a neuroid comprising at least one internalized MATRIGEL-absorbed microparticle.

In embodiments, step (II) occurs for up to 8 hours, up to 16 hours, up to 24 hours, up to 2 days, up to 3 days, up to 7 days, or up to 10 days and/or step (III) occurs after completion of step (II). In embodiments, the method further comprises culturing the neuroid comprising at least one internalized MATRIGEL-absorbed microparticle for at least 3 days, at least 7 days, at least 10 days, at least 14 days, at least 20 days, at least 30 days, at least 90 days, at least 200 days, at least 350 days or at least 500 days. The culturing of the plurality of cells in the absence of a microparticle provides improved rosette formation and area in the neuroid once contacted the one or more microparticles with MATRIGEL. The improved rosette formation and area is relative to a neuroid lacking a microparticle comprising MATRIGEL.

References relevant to neuronal organoids include in Velasco, et al., (2019). Individual brain organoids reproducibly form cell diversity of the human cerebral cortex. Nature, 570(7762), 523-527; Mansour, et al., (2018). An in vivo model of functional and vascularized human brain organoids. Nature Biotechnology, 36(5), 432-441; Kadoshima, et al., (2013). Self-organization of axial polarity, inside-out layer pattern, and species-specific progenitor dynamics in human ES cell-derived neocortex. Proceedings of the National Academy of Sciences, 110(50), 20284-20289; Lancaster & Knoblich (2014). Generation of cerebral organoids from human pluripotent stem cells. Nature Protocols, 9(10), 2329-2340; Watanabe, et al., (2017). Self-Organized Cerebral Organoids with Human-Specific Features Predict Effective Drugs to Combat Zika Virus Infection Resource Self-Organized Cerebral Organoids with Human-Specific Features Predict Effective Drugs to Combat Zika Virus Infection. CellReports, 21(2), 517-532; and Trujillo, et al., (2019). Complex Oscillatory Waves Emerging from Cortical Organoids Model Early Human Brain Network Development. Cell Stem Cell, October 3;25(4):558-569.e7. The contents of each of which are incorporated by reference in their entirety.

An aspect of the present disclosure is a neuroid produced by any herein-disclosed method.

### Cells

A method for producing a neuroid comprises (a) providing a plurality of cells in a culture vessel including at least one cell is a stem cell; (b) contacting the plurality of cells in the culture vessel with one or more microparticles; and (c) culturing the plurality of cells with the one or more microparticles, thereby producing a neuroid comprising at least one internalized microparticle.

In embodiments, the one or more microparticles comprises at least one functional agent. The stem cell may be an induced pluripotent stem cell (iPSC).

In some cases, the neuroids provided herein can be generated or derived from both somatic cells and precursor cells, such as stem cells. In general, a stem cell possesses the ability to differentiate into one, multiple or all types of cells in an organism. In some cases, a stem cell is unipotent; in some cases, a stem cell is multipotent or pluripotent. Pluripotent stem cells are self-renewing cells that are able to form teratomas when injected into an animal, and can contribute ectoderm, mesoderm, or endoderm tissues in a living organism. Examples of pluripotent stem cells are embryonic stem (ES) cells, embryonic germ stem (EG) cells, and iPSCs.

Neuroids of the present disclosure may include iPSCs. An iPSC is a pluripotent stem cell that is derived from a somatic cell, e.g., from a human subject. Like all pluripotent stem cells, iPSC can self-renew and give rise to cells of all three lineages (ectoderm, mesoderm, and endoderm). iPSCs have an ES cell-like morphology, growing as flat colonies with large nucleo-cytoplasmic ratios, defined borders and prominent nuclei. In addition, iPSCs express one or more key pluripotency markers, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42.

iPSCs used for generating neuroid can be created from a subject's somatic cells by contacting a somatic cell with certain reprogramming factors (e.g., Oct4, Sox2, Klf-4, Nanog, Lin28, and c-Myc). The somatic cells may be fibroblasts, adipocytes, stromal cells, and the like. Somatic cells or iPSCs can be obtained from cell banks, from normal donors, from individuals having a neurologic or psychiatric disease of interest. Methods for reprogramming somatic cells into iPSCs are well known in the art. See, for example, US20090191159; the contents of which is incorporated herein by reference in its entirety.

In some cases, the neuroids can be used to model a disease, e.g., any condition or disorder that damages or interferes with the normal function of a cell, tissue, or organ. In some embodiments, the diseases are neurological, neuropsychological, neuropsychiatric, neurodegenerative, or neuropsychopharmacological conditions. In some embodiments, the neurological conditions are neuropsychiatric diseases. Examples of diseases include neurological conditions, e.g., tuberous sclerosis, Autism, Rett Syndrome, Tuberous Sclerosis, Dravet Syndrome, Parkinson's Disease, Multiple sclerosis, Batten's disease or Alzheimer's Disease.

In embodiments, at least one neuroid in the set comprises a phenotype of a neurological condition.

In embodiments, at least one neuroid in the set is derived from cells obtained from a subject having a neurological condition and/or a plurality of cells in a neuroid comprises a genetic modification or mutation associated with a neurological condition.

Genes may be edited into the somatic cell's or the iPSC's genome prior to usage in forming a neuroid. The genes may be modified for a variety of purposes, e.g., to replace genes having a loss-of-function mutation, to induce a mutation, and to provide reporter genes. Alternatively, gene expression may be reduced in the cells, e.g., via introduction of antisense RNA, siRNA, miRNA, and shRNA. Drug resistance genes may be introduced to provide selective advantage to cells during culturing. Various techniques may be used to introduce nucleic acids into the target cells, e.g., electroporation, calcium precipitated DNA, fusion, transfection, lipofection, infection, and the like.

Cells used to generate neuroid may be modified to carry disease-associated genotypes. For examples disease-associated or disease-causing genotypes can be generated (gene-edited) in healthy cells through targeted genetic manipulation (CRISPR/CAS9 or TALENs) or cells can be derived from individual patients that carry a disease-related genotype or are diagnosed with a disease. Gene edited cell lines can share the same genetic background as their corresponding, non-edited cells, thereby reducing variability associated with line-line differences in genetic background.

In some cases, neural diseases with less defined or without genetic components can be studied using neuroids of the presented disclosure, e.g., conditions relating to neurodevelopmental and neuropsychiatric disorders and neural diseases correlated with genetic or genomic alterations can be modeled.

Illustrative genetic alterations include point mutations in genes such as NLGN1/3/4, NRXN1/4, SHANK1/2/3, GRIN2B/A, FMR1, or CHD8 that represent risk alleles for autism spectrum disorders, point mutations in or deletions of genes such as CACNA1C, CACNB2, NLGN4X, LAMA2, DPYD, TRRAP, MMP16, NRXN1 or NIPAL3 that are associated with schizophrenia or autism spectrum disorders (ASD), a triplet expansion in the HTT gene that cause to Huntington's disease (HD), monoallelic mutations in genes such as SNCA, LRRK2 and biallelic mutations in genes such as PINK1, DJ-1, or ATP13A2 that predispose to Parkinson disease (PD), single nucleotide polymorphisms (SNPs) in genes such as ApoE, APP, and PSEN1/2 that confer risks for developing Alzheimer's disease (AD) and other forms of dementia, as well as SNPs in genes such as CACNA1C, CACNB3, ODZ4, ANK3 that are associated with bipolar disease (BP); Angelman (UBE3A), Rett (MEPC2), Tuberous sclerosis (TSC1/2). Genomic alterations include copy number variations (CNVs) such as deletions or duplications of 1q21.1, 7q11.23, 15q11.2, 15₈13.3, 22q11.2 or 16p11.2, 16p13.3 that are associated with ASD, schizophrenia, intellectual disability, epilepsy; trisomy 21 and Down Syndrome, Fragile X syndrome caused by alteration of the FMR1 gene.

In some cases, the neuroids are derived from lineage-committed stem cells, which are generally multipotent stem cells that give rise to cells of a specific lineage. Examples of lineage-committed stem cells include mesodermal stem cells. In some cases, the lineage-committed stem cells are tissue-specific stem cells, such as multipotent stem cells that reside in a particular tissue and are capable of clonal regeneration of cells of the tissue in which they reside. Lineage-committed stem cells may be neuronal stem cells that are able to reconstitute all neuronal/glial lineages. Progenitor cells differ from tissue-specific stem cells in that they typically do not possess extensive self-renewal capacity, and often can only regenerate a subset of the lineages in the tissue from which they derive, for example only neurons or glia in the nervous system.

In some embodiments, stem cells for generating neuroids may be derived from a mammalian tissue, e.g., brain tissue. The cells are isolated from the mammalian tissue and cultured for a time sufficient to enrich for stem cells, form neuroids, and/or induce differentiation of cells in a neuroid.

In embodiments, pluripotent stem cells (e.g., iPSCs) are cultured to form a cell aggregate and the pluripotent stem cells are induced to differentiate into a tissue (e.g., neural tissue). The tissue may contain at least one of pluripotent stem cells, multipotent stem cells, progenitor cells, terminally differentiated cells, endothelial cells, endothelial progenitor cells, immortalized cell lines, or primary cells.

A pluripotent stem cell may be cultured in a condition that results in an enriched population of specific multipotent stem cells (e.g., neural progenitor cells). In some examples, a pluripotent stem cell may be cultured in a condition that results in an enriched population of specific multipotent stem cells (e.g., neural progenitor cells) to form a neuroid by directed differentiation. The term directed differentiation refers to the culture of pluripotent or multipotent stem cells in conditions that preferentially encourage the differentiation of the stem cell into a specific, more differentiated state. Alternatively, a multipotent stem cell (e.g., neural stem cell) may be directly differentiated into a more differentiated state (e.g., a neuron, astrocyte, and oligodendrocyte). The culture condition that results in an enriched population of specific multipotent stem cells to form a tissue may occur by transdifferentiation. The term transdifferentiation refers to the conversion of one cell type that may be a multipotent or unipotent stem cell, or a terminally differentiated mature cell phenotype to a different cell type that may be a different multipotent or unipotent stem cell, or a terminally differentiated mature cell phenotype. For example, a neural stem cell, a radial glia, or a neuron may be transdifferentiated into an endothelial cell.

In some cases, stem cells can be converted to differentiated cells (e.g., neuronal cells) using a neuronal reprogramming system. In some embodiments, pluripotent cells including, without limitation, embryonic stem cells, induced pluripotent stem cells, can be converted into neuronal cells by contacting the pluripotent cell with a neuronal reprogramming system comprising one or more factors selected from an Ascl agent, a Ngn agent, a Brn agent, a NeuroD agent, a Mytl agent, an Olig agent or a Zic agent. In certain embodiments, neuron reprogramming factors are combination of an Ascl agent, a Brn agent, a NeuroD agent, and a Myt1 agent, which combinations of interest include without limitation, AscM , Brn2, Myt11 and NeuroDL. By agent is meant either the named protein (e.g., Ngn) or a nucleic acid comprising the named protein (e.g., ngn mRNA). Many of the agents described herein are transcription factors.

In embodiments, a neuroid comprises one or more cells expressing a factor capable of promoting neuroid growth or patterning. In some cases, the cells of a neuroid express a morphogen, either naturally or due to genetic engineering. In some cases, the cells expressing the factor are coated on the microparticle or the cells expressing the factor can act as support cells for the developing neuroid, e.g., cells that are non-neural in nature/fate.

### Kits

In an aspect, the present disclosure provides a kit to make a neuroid from a single cell or from a plurality of cells. The kit may contain microparticles, functional agents for microparticles, culturing medium (e.g., differentiation medium and/or a neuroid medium), and culturing vessels. The kit may also comprise useful supplements (growth factors, antibiotics, hormones, vitamins, amino acids, and combinations thereof) to be added to a culturing medium. The culturing vessel in a kit may comprise topological features (e.g., cavities or microwells) to enable production of consistent neuroids of similar shape and size. The kit may comprise biocompatible matrix materials, e.g., Matrigel™. The kit may further contain a set of instructions to perform methods of making a neuroid.

### DEFINITIONS

The terminology used herein is for the purpose of describing particular cases only and is not intended to be limiting.

Unless defined otherwise, all technical and scientific terms used herein have the same meaning as commonly understood by one of ordinary skill in the art. Although any methods and materials similar or equivalent to those described herein can be used in the practice or testing of the present disclosure, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As used herein and in the appended claims, the singular forms “a”, “an”, and “the” include plural referents unless the context clearly dictates otherwise. Thus, for example, reference to “the induced pluripotent stem cells” includes reference to one or more induced pluripotent stem cells and equivalents thereof known to those skilled in the art, and so forth.

The terms “contain,” “containing,” “including”, “includes”, “having”, “has”, “with”, or variants thereof as used in either the present disclosure and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

Whenever the term “at most about” or “at least about” precedes the first numerical value in a series of two or more numerical values, the term “at most about” or “at least about” applies to each of the numerical values in that series of numerical values. For example, at most about 3, 2, or 1 is equivalent to at most about 3, at most about 2, or at most about 1.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

The use of “or” throughout the disclosure encompasses multiple variations of any grouping of a stated composition or method. For example, the phrase “A or B” may be nonexclusively read as meant to include “A but not B”, “B but not A”, and “A and B” unless otherwise specified or indicated by context.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The terms “cell culture” or “culture,” as used herein, general refer to the maintenance of cells in an artificial, in vitro environment. It is to be understood, however, that the term “cell culture” is a generic term and may be used to encompass the cultivation not only of individual cells, but also of tissues or organs. The term “culture system” is used herein to refer to the culture conditions in which the subject explants are grown that promote prolonged tissue expansion with proliferation, multilineage differentiation and recapitulation of cellular and tissue ultrastructure.

The terms “treatment”, “treating”, “treat” and the like are used herein to generally refer to obtaining a desired pharmacologic and/or physiologic effect. The effect may be therapeutic and involve a partial or complete stabilization or cure for a disease and/or adverse effect attributable to the disease. For example, the effect may involve the reduction or elimination of one or more symptoms of a disease or disorder. In some cases, the effect may be prophylactic and may completely or partially prevent a disease or symptom thereof. The tern “treatment” as used herein encompasses any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; or (c) relieving the disease symptom, e.g., causing regression of the disease or symptom. The terms “individual,” “subject,” “host,” and “patient,” are used interchangeably herein and refer to any mammalian subject for whom diagnosis, treatment, or therapy is desired, particularly humans. The term can also encompass non-human mammals such as apes, monkeys, chimpanzees, mice, rabbits, dogs, cats.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue.

The term “contacting”, as used herein, carries its normal ordinary definition and also can refer to the placing of candidate cells or candidate agents into the pro-organ culture as described herein.

Any aspect or embodiment described herein can be combined with any other aspect or embodiment as disclosed herein.

### EXAMPLES

### Example 1

### Preparation of Functional Agent-Coated Microparticles for Neuroid Generation

The following example describes the generation of functional agent-coated polystyrene microparticles (Sigma-Aldrich, 16435). Two ml of polystyrene microparticles (200 μM in diameter) were aliquoted into two separate Eppendorf tubes (1 ml each). The liquid from the tubes was aspirated and the microparticles were subsequently incubated in 10% HCl solution in deionized (DI) water for thirty minutes accompanied by gentle shaking. The HCl solution was aspirated and the microparticles were incubated in 70% isopropanol for fifteen minutes with gentle shaking. The Isopropanol solution was aspirated and microparticles were washed with 1 ml sterile phosphate buffered saline (PBS). The microparticles were centrifuged followed by aspiration of PBS. The microparticles were washed with PBS three times in total.

Microparticles from the previous step were aliquoted and incubated at room temperature in one of the following protein solutions: (1) 20 μg/ml fibronectin (FN Sigma-Aldrich, F2006-1mg), (2) laminin (LN, Sigma-Aldrich, L2020-1mg), or (3) collagen IV (Col IV, Advanced Biomatrix, 5022-5 mg). Microparticles were gently rocked in protein solution for one hour at room temperature and then kept overnight at 4° C. Excess or unbound protein was removed by three washes with sterile PBS. The microparticles comprising a functional agent (i.e., protein-coated polystyrene beads) were suitable use in forming neuroids incorporating microparticles.

### Example 2

### Generation of Neuroids Incorporating Polystyrene Microparticles Comprising a Functional Agent

Protein-coated microparticles from Example 1 were used to generate neuroids. Protein-coated microparticles were resuspended evenly in sterile PBS. Eight μl of the protein-coated microparticles in PBS was aliquoted in each well of a 96-well, U-bottom plate with a non-adhesive surface. Each well contained approximately 3-20 microparticles. 10,000 iPSCs from different cell lines (Ex5, Ex20, Ex24, or Sys15c1 cell lines), were seeded immediately into each well containing the protein-coated microparticles in 150 μL of culture media. Plates comprising microparticles and cells were centrifuged at 120 g for one minute. The plates were incubated for three days without disruption at 37° C., 5% CO2, and 20% O₂. On the third day, 75% of the media per well was aspirated and replaced with 150 μL of fresh culture media. The plates were then incubated at 37° C., 5% CO₂, and 40% O₂. By day 3 or day 4, all added microparticles had been internalized into the developing neuroid.

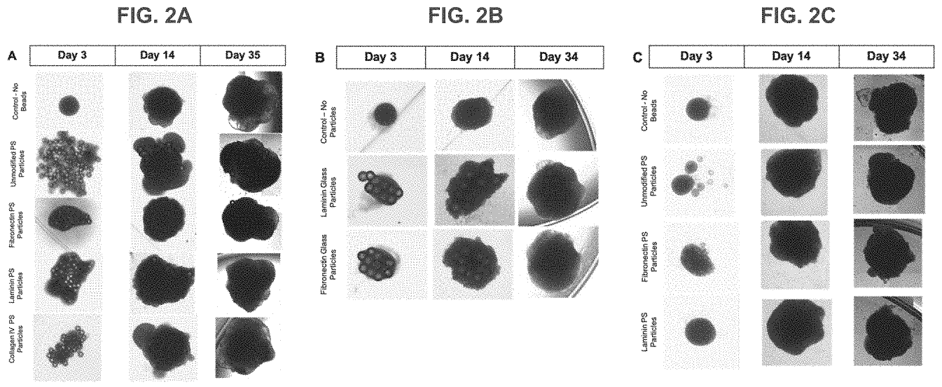

FIG. 2A to FIG. 2C show representative bright field images of control neuroids cultured without internalized microparticles and neuroids incorporated with internalized microparticles; shown at day 3, day 14 and day 34 of in vitro growth. FIG. 2A shows representative bright field images of neuroids that have incorporated polystyrene microparticles (with or without a functional agent); shown are the neuroids at day 3, day 14 and day 34 of in vitro growth. FIG. 2B shows representative bright field images of neuroids that have incorporated glass microparticles (with either laminin or fibronectin as functional agent); shown are the neuroids at day 3, day 14 and day 34 of in vitro growth. FIG. 2C shows representative bright field images of neuroids that have incorporated agarose microparticles (with or without a functional agent); shown are the neuroids at day 3, day 14 and day 34 of in vitro growth. Neuroids shown in these figures comprise cells from the Sy15c1 or Ex22 cell line.

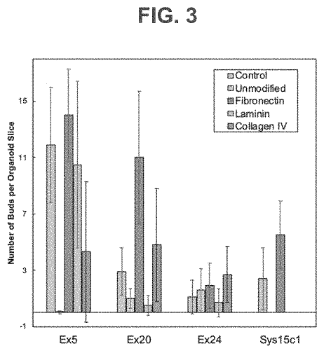

FIG. 3A shows a comparison of the quantification of rosette formation in neuroids incorporated with polystyrene particles derived from one of four cell lines (Ex5, Ex20, Ex24 or Sys15c1). Neuroids cultured with polystyrene microparticles coated with fibronectin showed an increased number of cortical rosettes per neuroid slice.

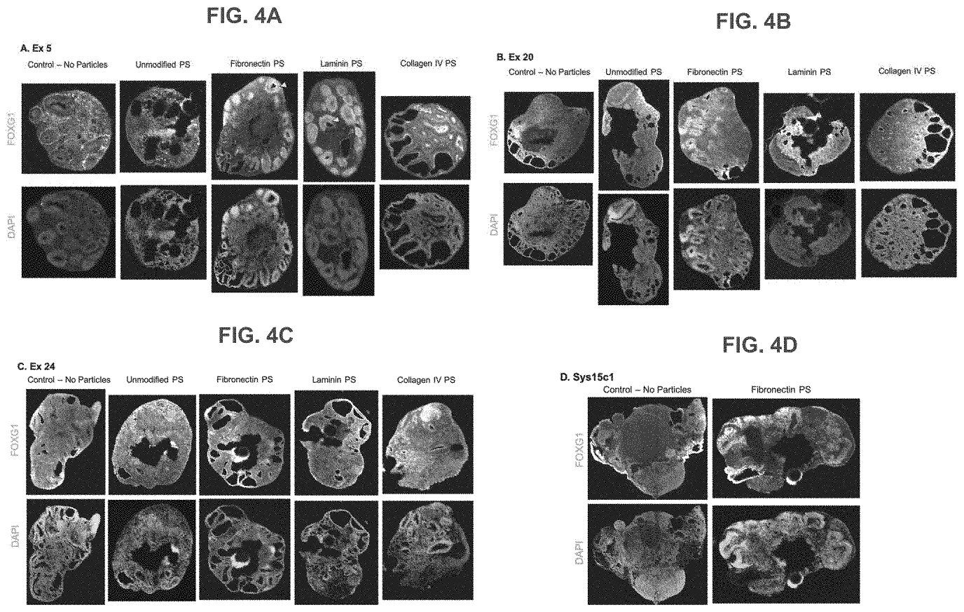

FIG. 4A to FIG. 4D shows immunofluorescence images of FOXG1 and DAPI staining of neuroid slices (at day 32-35 of growth) derived from one of the Ex5, Ex20, Ex24 or Sys15c1 cell lines. FIG. 4A shows immunofluorescence images for neuroids derived from cells of the Ex5 cell line that comprise internalized fibronectin-, laminin-, or collagen IV-coated polystyrene particles. Neuroids from the Ex5 cell line, grown with polystyrene particles coated with fibronectin, laminin or collagen shown an increased expression of FOXG1 compared to neuroids that do not comprise internalized microparticles. FIG. 4B shows immunofluorescence images for neuroids derived from cells of the Ex20 cell line that comprise internalized fibronectin-, laminin-, or collagen IV-coated polystyrene particles. Neuroids from the Ex20 cell line, grown with internalized polystyrene particles coated with fibronectin shown an increased expression of FOXG1 compared to neuroids that do not comprise internalized microparticles or neuroids comprising microparticles uncoated with fibronectin. FIG. 4C shows immunofluorescence images of neuroids derived from cells of the Ex24 cell line that were cultured with internalized fibronectin-, laminin-, or collagen IV-coated polystyrene particles. Neuroids from the Ex24 cell line did not show increased expression of FOXG1 for any of the conditions compared to neuroids that do not comprise internalized microparticles or neuroids comprising uncoated microparticles. FIG. 4D shows immunofluorescence images of neuroids derived from cells of the Sys15C1 cell line that were cultured with internalized fibronectin-, laminin-, or collagen IV-coated polystyrene particles. Neuroids from the Sys15c1 cell line, grown with polystyrene particles coated with fibronectin shown an increased expression of FOXG1 compared to neuroids that do not comprise internalized microparticles or neuroids comprising microparticles uncoated with fibronectin.

In the immunofluorescence experiments, neuroids were isolated from their culture medium and washed in a saline solution. The neuroids were fixed in a 4% (v/v) paraformaldehyde solution for two hours; then, washed and incubated in 30% sucrose overnight at 4° C. The neuroids were washed, embedded in OCT, and flash frozen on dry ice. The embedded neuroids were stored at −80° C. until cryosectioning. Cryosections were labeled with primary antibodies and fluorescent secondary antibodies. Labeled cryosections were imaged and the presence or absence of fluorescent signals were detected.

### Example 3

### Generation of Neuroids Comprising Functionalized Agarose Microparticles

4% (w/v) agarose beads (Agarose Bead Technologies, 50-150 μm diameter, #A-1040S-500) were centrifuged in 2 mL Eppendorf tubes and the storage liquid was aspirated. The agarose beads were sterilized in 70% isopropanol (IPA) for one hour. The IPA was aspirated and the agarose beads were incubated for at least six hours in sterile PBS with gentle agitation to form a slurry. The slurry was centrifuged and the PBS was aspirated. The slurry formation and aspiration steps were repeated two to three times.

The agarose beads were twice rinsed in 0.01 M Tris Buffer (pH 8.5) followed by incubation in 2 mg/mL dopamine hydrochloride (Sigma-Aldrich, #H8502) in Tris Buffer (filtered through a 0.2 μm filter immediately before use). Incubation was for about two hours with gentle agitation. The resulting agarose-PDA microparticles were twice washed with sterile PBS and stored at 4° C. The day before use, agarose-PDA microparticles were incubated in cold 5% (v/v) MATRIGEL in PBS and stored on ice at 4° C. Other agarose-PDA microparticles were incubated in 20 μg/mL fibronectin in PBS or 20 μg/mL laminin in PBS with gently agitation for two hours at room temp and then incubated on ice at 4° C. Control agarose-PDA microparticles lacking a functional agent was also prepared.

Agarose-PDA microparticles with a functional agent or control microparticles (which lacked a functional agent) were removed from ice and warmed to room temperature. Microparticles were twice washed in PBS. Beads diluted so that 10 μL of a slurry included between about ten to twenty beads. Ten μl of the diluted microparticles was aliquoted in each well of a 96-well, U-bottom plate with a non-adhesive surface. 10,000 iPSCs from different cell lines (Ex5, Ex20, Ex24, or Sys15c1 cell lines), were seeded immediately into each well containing the microparticles in 150 μL of culture media. Plates comprising microparticles and cells were centrifuged at 120g for one minute.

The plates were incubated for three days without disruption at 37° C., 5% CO₂, and 20% O₂. On the third day, 75% of the media per well was aspirated and replaced with 150 μL of fresh culture media. The plates were then incubated at 37° C., 5% CO₂, and 40% O₂. By day 3 or day 4, all added agarose-PDA microparticles comprising fibronectin or laminin had been internalized into the developing neuroid. Control microparticles, which lacked a protein functional agent, were not initially internalized by developing neuroids. On day 10, a 5% MATRIGEL solution was added to the control cultures and developing neuroids immediately began internalizing the “control” microparticles which, by this point, had absorbed the MATRIGEL. These microparticles were fully internalized within two days (by day 12).

Labeled cryosections were prepared imaged as described in Example 2.

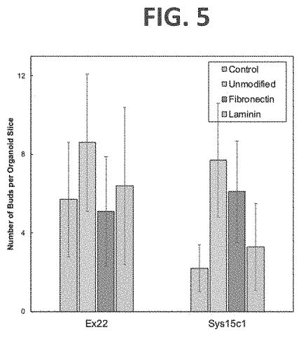

FIG. 5A shows immunofluorescence images comparing the levels of FOXG1 and DAPI expression in neuroid slices (at day 32-35 of in vitro growth) derived from cells of the Sys15C1 cell line that were cultured with internalized agarose microparticles coated with fibronectin or laminin. Neuroids comprising agarose microparticles lacking fibronectin or laminin showed an increase in the number of cortical rosettes per neuroid slide when compared to neuroids comprising fibronectin or laminin coated microparticles.

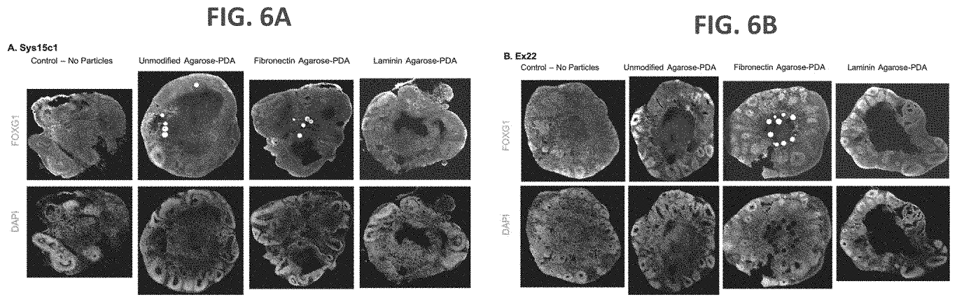

FIG. 6A and FIG. 6B show immunofluorescence images displaying the levels of FOXG1 and DAPI expression in pro-organ slices (at day 34 of in vitro growth) derived from cells of the Sys15C1 or Ex22 cell lines. FIG. 6A shows immunofluorescence images for neuroids derived from cells of the Sys15C1 cell line that comprise agarose particles coated with fibronectin or laminin. FIG. 6B shows immunofluorescence images for neuroids derived from cells of the Ex22 cell line that comprise agarose particles coated with fibronectin or laminin. Neuroids comprising agarose microparticles lacking fibronectin or laminin showed increased expression of FOXG1 slide when compared to neuroids comprising fibronectin or laminin coated microparticle.

### Example 4

### Generation of Neuroids Incorporating Magnetic Microparticles

Magnetically-active neuroids were generated by seeding iPSCs with magnetically-active 50 μm microparticles in a non-adhesive U-bottom microwell plate.

A 20% slurry of Promega Magne Protein A microparticles (300 μl) was centrifuged, the solution aspirated, and then washed in 70% isopropanol for fifteen minutes. The microparticles were washed three times with sterile PBS and then diluted to 1.3 ml (a 5.4% slurry). Ten μl of the slurry was added to wells of a microwell plate. Ten thousand cells were plated per well followed by centrifugation at 300 g for one minute. Cells were incubated at 37° C.

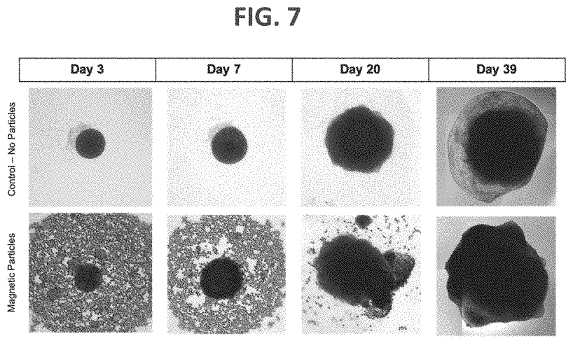

As shown in FIG. 7, cells adhered to the particles and ingested them into an embryonic body (EB). On day seven of the neuroid culture, the extracellular matrix protein mixture, MATRIGEL, was added as a liquid to the microwell. With increased temperature, the MATRIGEL solidified into a gel, encasing the neuroid and the remaining magnetic particles surrounding the developing neuroid (See, FIG. 7). Over time, the neuroid matures and the remaining magnetic particles are internalized into the neuroid.

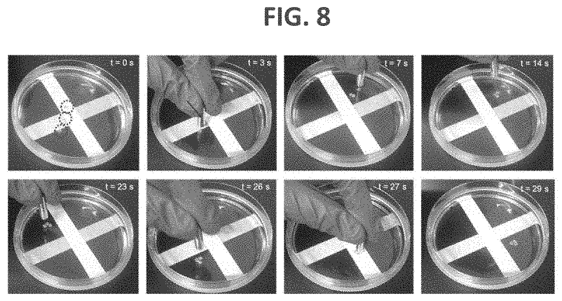

Neuroids comprising magnetic microparticles were magnetically manipulated in liquid environments. As shown in FIG. 8, three neuroids, suspended in PBS, were transferred as a group around a Petri dish in under 30 seconds using an external magnet. The magnet did not make contact with the PBS solution.

Including a large initial quantity of magnetic microparticles permitted the resulting neuroid to be magnetically manipulated over time (e.g., up to Day 41); this was surprising given the considerable increases in tissue mass and volume that the neuroid accumulates.

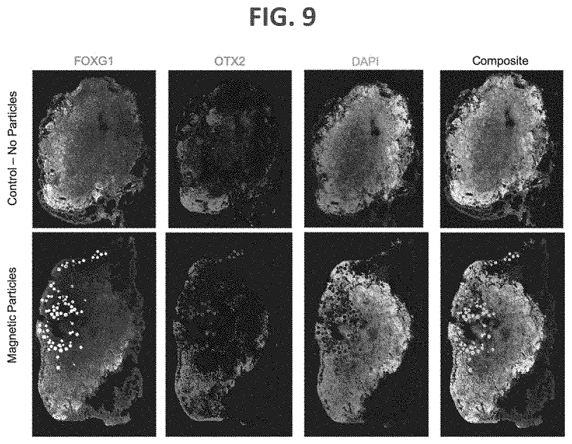

To ensure the magnetic microparticles do not disrupt neuroid formation, immunofluorescence images of the magnetic neuroids were taken and compared to controls (which lack microparticles). As seen in FIG. 9, neuroids in both groups show neural rosette formation and expression of the neuronal markers, FOXG1 and OTX2. This indicates that the magnetic microparticles do not disrupt neuroid growth or function. The small neural rosettes on the edges also indicate organized tissue that is a key feature of neuroid classification which distinguish the tissue from spheroids (which are often formed from cancer cell lines or tumor biopsies as freely floating cell aggregates in ultra-low attachment plates).

In alternate experiments, a magnetic bead is coated with polydopamine (PDA) in addition to or in the place of Protein A.

### Example 5

### Use of Neuroids for Screening Candidate Agents

In this example, neuroids are used for screening agents, e.g., that affect neural development/function and/or treat neurological disease.

Neuroids are prepared according to any herein disclosed method. Preferably, neuroids are prepared in a microwell or at an air-liquid interface since these methods often produce uniform neuroids that improve accuracy and reproducibility of screening assays, in part, due to a reduction in variability in neuroid size, shape, and cell density.

Here, a candidate agent is contacted with a neuroid and a phenotypic change, if any, is detected in one or more neuroids or by a cell thereof. When the agent is capable of producing a phenotypic change in a neuroid or a cell thereof, then this agent may be useful as a therapeutic, e.g., for treating a neurological disease. A detected phenotypic change may be a modification in the neuroid's longest dimension, cell type, shape, viability, number of cells, oscillatory electrical activity, calcium activity, signaling pathway activation, protein-protein interactions, phosphorylation, ubiquitination, sumolyation, RNA expression, protein expression, genotype, and/or epigenomic type.

### Example 6

### Use of Neuroids for Modeling Neural Development

In this example, neuroids are used to model neural development.

Neuroids of the present disclosure properly model native nervous system tissue, express a range of neural markers (including brain region specific genetic and molecular markers), and comprise cellular diversity and cytoarchitecture consistent with in vivo nervous system tissue. Also, neuroids of the present disclosure also possess functions/activity that approximate in vivo neural system tissue and/or organs and/or specific regions of the mammalian nervous system.

For example, the neuroid may model cortical tissue and express neural markers characteristic of cortical neurons (e.g., FOXG1). Such cortical neuroids may be used to study neural circuit formation. Here, two cortical neuroids may be contacted and a neural circuit, which approximates a circuit present in the mammalian cortex, can be studied. A cortical neuroid and a neuroid of a different type (e.g., a cerebellar neuroid or a spinal cord neuroid) may be contacted.

A spinal cord neuroid may be contacted with a pro-organ comprising non-neural cells, e.g., muscle cells. Such a combination approximates connections that nervous system cells/tissues make with other cells/tissues of the body. For example, assays relating to contacts between neural tissue and the enervated skeletal muscle (e.g., via nicotinergic nerve endings at skeletal neuromuscular junctions (NMJ)) can be conducted.

A functional neural circuit between two neuroids and/or between a neuroid and a non-neural pro-organ can comprise fluid, physical, synaptic, and/or electrical communications. Functional neural circuit may be characterized by electrical activity patterns within a network of cells/neuroids.

The circuit models of this Example may be combined with a screening assay, as described in the disclosure. As an example, the effects of candidate agent on a functional neural circuit may be tested. Here, first a functional circuit is established; then, two neuroids comprising the neural circuit or a neuroid and a non-neural pro-organ are contacted with the candidate agent. Any change in activity in the neural circuit suggests that the agent may be useful as a therapeutic, e.g., for treating a neurological disease.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

It is to be understood that this disclosure is not limited to particular compositions and methods described, as such may, of course, vary. It is also to be understood that the terminology used herein is for the purpose of describing particular embodiments only, and is not intended to be limiting, since the scope of the present disclosure will be limited only by the appended claims.

### INCORPORATION BY REFERENCE

All publications, patents, and patent applications mentioned in this Specification are herein incorporated by reference in their entireties to the same extent as if each individual publication, patent, or patent application was specifically and individually indicated to be incorporated by reference.

1-98. (canceled)

99. A set of neuroids wherein each neuroid in the set comprises one or more internalized microparticles, and wherein each neuroid in the set expresses a neural marker that is indicative of the presence of at least two separate brain regions, and wherein the one or more internalized microparticles are coupled to one or more functional agents selected from fibronectin, laminin, collagen IV, and MATRIGEL.

100. The set of neuroids of claim 99, wherein the one or more internalized microparticles comprises a hydrogel comprises a polymer, glass, hydrogel, plastic, silica, ceramic, or magnetic sub stance.

101. The set of neuroids of claim 99, wherein the one or more internalized microparticles are coated with protein A or polydopamine (PDA).

102. The set of neuroids of claim 99, wherein the one or more internalized microparticles encapsulates one or more functional agents.

103. The set of neuroids of claim 102, wherein the one or more functional agents modulates cell adhesion, provides spatial control of cell adhesion, cell growth, cell proliferation, cell organization, cell differentiation, cell repair, or cell regeneration in the neuroid.

104. The set of neuroids of claim 99, wherein a neuroid in the set further comprises at least one additional microparticle comprising one or more functional agents selected from the group consisting of: a polypeptide, a polysaccharide, a small molecule, a nucleic acid, an imaging agent, MATRIGEL, hydrogel, and a sensor, or any combination thereof.

105. The set of neuroids of claim 99, wherein the neural marker is selected from a cortical marker, a hippocampal marker, a cerebellar marker, a retinal marker, a midbrain marker, a spinal cord marker, or a brain stem marker.

106. The set of neuroids of claim 99, wherein each neuroid in the set of neuroids are distinct.

107. The set of neuroids of claim 99, wherein at least one neuroid in the set comprises a phenotype of a neurological condition, wherein the phenotype relates to one or more of: size/shape of the neuroid, cell type, viability, number of cells, electrical activity, calcium activity, RNA expression, protein expression, genotype, or epigenomic type.

108. The set of neuroids of claim 99, wherein at least one neuroid in the set is derived from cells obtained from a subject having a neurological condition.

109. The set of neuroids of claim 99, wherein a plurality of cells in at least one neuroid in the set comprises a genetic modification or mutation associated with a neurological condition.

110. The set of neuroids of claim 99, wherein the one or more internalized microparticles provides an at least 10% increase in the expression of a neural marker by cells of the one or more neuroids, wherein the increase is relative to a neuroid prepared in an identical manner and with identical cells but that does not comprise the one or more internalized microparticles.

111. The set of neuroids of claim 99, wherein at least one neuroid in a set comprises a concentration gradient for the functional agent, wherein the concentration gradient originates at an internalized microparticle.

112. The set of neuroids of claim 99, wherein at least one neuroid in the set is capable of generating neuronal activity.

113. The set of neuroids of claim 99, wherein at least one neuroid in the set comprises a functional neural circuit between two cells in a neuroid.

114. The set of neuroids of claim 99, wherein a plurality of neuroids in the set are in fluid, physical, synaptic, or electrical communication with other neuroids in the set.

115. The set of neuroids of claim 99, wherein the set comprises at least 2, 3, 4, 5, 6, 7, 8, 9, 10, 50, 100, 300, 500, 1,000, 10,000, 100,000, or 1,000,000 neuroids, e.g., 96, 384, and 1536 neuroids.

116. The set of neuroids of claim 99, wherein the neuroids are spatially dispersed in a two-dimensional array.

117. The set of neuroids of claim 99, wherein a plurality of neuroids in the set comprise fusion neuroids including two or more previously-distinct neuroids and/or a fusion between a neuroid and a non-neuroid tissue, e.g., a native neural tissue, a blood vessel tissue, and glial tissue, including microglia.

118. The set of neuroids of claim 99, wherein the one or more internalized microparticles provides improved rosette formation and area in the neuroid relative to a neuroid prepared in an identical manner and with identical cells but that does not comprise the one or more internalized microparticles.

120. A method of producing a neuroid comprising at least one internalized microparticle, the method comprising:
(i) providing a plurality of cells in a culture vessel, wherein at least one cell is a stem cell;
(ii) contacting the plurality of cells in the culture vessel with one or more microparticles lacking a functional agent;
(iii) culturing the plurality of cells with the one or more microparticles lacking a functional agent;
(iv) contacting the one or more microparticles with MATRIGEL, thereby allowing the microparticles to absorb the MATRIGEL; and
(v) further culturing the plurality of cells with the one or more microparticles that have absorbed MATRIGEL, thereby producing a neuroid comprising at least one internalized MATRIGEL-absorbed microparticle.

121. A method of manipulating one or more neuroids in a culture vessel, the method comprising: (a) providing one or more neuroids comprising at least one internalized microparticle comprising a magnetic substance; and (b) applying an external magnetic field to the one or more neuroids, thereby manipulating the one or more neuroids in the culture vessel.
